SEQUENCE LISTING (254 sequences)

SEQ ID NO: 1 - protein, 6 aa, Mus musculus
GRLLYR

SEQ ID NO: 2 - protein, 17 aa, Mus musculus
ADPLGLEAEKDGVPVFK

SEQ ID NO: 3 - protein, 16 aa, Mus musculus
ILPNVPEVEDSTDFFK

SEQ ID NO: 4 - protein, 21 aa, Mus musculus
GSASSALELTEEELATAEAVR

SEQ ID NO: 5 - protein, 14 aa, Mus musculus
LFVEDSIHDQFVQK

SEQ ID NO: 6 - protein, 8 aa, Mus musculus
FLFPEGIK

SEQ ID NO: 7 - protein, 13 aa, Mus musculus
HGSIIYHPSLLPR

SEQ ID NO: 8 - protein, 16 aa, Mus musculus
GVVNILPGSGSLVGQR

SEQ ID NO: 9 - protein, 14 aa, Mus musculus
DTNHGPQNHEAHLR

SEQ ID NO: 10 - protein, 11 aa, Mus musculus
TDVAAPFGGFK

SEQ ID NO: 11 - protein, 12 aa, Mus musculus
EESFGPIMIISR

SEQ ID NO: 12 - protein, 15 aa, Mus musculus
ANATEFGLASGVFTR

SEQ ID NO: 13 - protein, 16 aa, Mus musculus
RPQPEEGATYEGIQKK

SEQ ID NO: 14 - protein, 12 aa, Mus musculus
FADGDVDAVLSR

SEQ ID NO: 15 - protein, 15 aa, Mus musculus
IQGATIPINQARPNR

SEQ ID NO: 16 - protein, 13 aa, Mus musculus
LQAGTVFVNTYNK

SEQ ID NO: 17 - protein, 15 aa, Mus musculus
RPQPEEGATYEGIQK

SEQ ID NO: 18 - protein, 12 aa, Mus musculus
DLGEAALNEYLR

SEQ ID NO: 19 - protein, 11 aa, Mus musculus
AGLILFGNDDR

SEQ ID NO: 20 - protein, 11 aa, Mus musculus
THVGMSIQTFR

SEQ ID NO: 21 - protein, 9 aa, Mus musculus
MMPASQFFK

SEQ ID NO: 22 - protein, 16 aa, Mus musculus
ECDVLPDDTVSTLYNR

SEQ ID NO: 23 - protein, 9 aa, Mus musculus
LIAEGTAPR

SEQ ID NO: 24 - protein, 10 aa, Mus musculus
DIPVDSPELK

SEQ ID NO: 25 - protein, 12 aa, Mus musculus
ENEFFIVTQTCK

SEQ ID NO: 26 - protein, 21 aa, Mus musculus
EVLGHSIAQLNAENDHPFYYK

SEQ ID NO: 27 - protein, 10 aa, Mus musculus
GNLFMDINNK

SEQ ID NO: 28 - protein, 7 aa, Mus musculus
QFNPGVK

SEQ ID NO: 29 - protein, 9 aa, Mus musculus
RPPGFSPFR

SEQ ID NO: 30 - protein, 9 aa, Mus musculus
SGNQYMLHR

SEQ ID NO: 31 - protein, 11 aa, Mus musculus
TDGSPTFYSFK

SEQ ID NO: 32 - protein, 8 aa, Mus musculus
YVIEFIAR

SEQ ID NO: 33 - protein, 12 aa, Mus musculus
AYYHLLEQVAPK

SEQ ID NO: 34 - protein, 16 aa, Mus musculus
FSLVGIAGQDLNEGNR

SEQ ID NO: 35 - protein, 17 aa, Mus musculus
GDEEGIPAVVIDMSGLR

SEQ ID NO: 36 - protein, 11 aa, Mus musculus
IGLFADIELSR

SEQ ID NO: 37 - protein, 8 aa, Mus musculus
ISFDEFIK

SEQ ID NO: 38 - protein, 9 aa, Mus musculus
LSPEELLLR

SEQ ID NO: 39 - protein, 13 aa, Mus musculus
MINLSVPDTIDER

SEQ ID NO: 40 - protein, 9 aa, Mus musculus
NEALIALLR

SEQ ID NO: 41 - protein, 10 aa, Mus musculus
QFVTATDVVR

SEQ ID NO: 42 - protein, 8 aa, Mus musculus
VNKPPYPK

SEQ ID NO: 43 - protein, 14 aa, Mus musculus
VYALPEDLVEVNPK

SEQ ID NO: 44 - protein, 7 aa, Mus musculus
YAISMAR

SEQ ID NO: 45 - protein, 14 aa, Mus musculus
YTLNILEDIGGGQK

SEQ ID NO: 46 - protein, 9 aa, Mus musculus
APLPLGHIK

SEQ ID NO: 47 - protein, 13 aa, Mus musculus
ASMHPVTAMLVGR

SEQ ID NO: 48 - protein, 15 aa, Mus musculus
DVDPDVAYSSIPYEK

SEQ ID NO: 49 - protein, 10 aa, Mus musculus
ELVALMSAIR

SEQ ID NO: 50 - protein, 7 aa, Mus musculus
FTRPLFK

SEQ ID NO: 51 - protein, 14 aa, Mus musculus
GSPMEISLPIALSK

SEQ ID NO: 52 - protein, 9 aa, Mus musculus
MKFTRPLFK

SEQ ID NO: 53 - protein, 16 aa, Mus musculus
MQEVYNFNAINNSEIR

SEQ ID NO: 54 - protein, 13 aa, Mus musculus
SANEFSETESMLK

SEQ ID NO: 55 - protein, 8 aa, Mus musculus
SFLYSHFK

SEQ ID NO: 56 - protein, 13 aa, Mus musculus
SHDQAVHTYQEHK

SEQ ID NO: 57 - protein, 10 aa, Mus musculus
TFGESHPFTK

SEQ ID NO: 58 - protein, 18 aa, Mus musculus
TLTGTAALTVQSQEENLR

SEQ ID NO: 59 - protein, 7 aa, Mus musculus
TQHLHLR

SEQ ID NO: 60 - protein, 9 aa, Mus musculus
VVINGQEVK

SEQ ID NO: 61 - protein, 10 aa, Mus musculus
YTLGESQGYK

SEQ ID NO: 62 - protein, 16 aa, Mus musculus
ADNFEYSDPVDGSISK

SEQ ID NO: 63 - protein, 9 aa, Mus musculus
DLEALMLDR

SEQ ID NO: 64 - protein, 8 aa, Mus musculus
ELLSGPNR

SEQ ID NO: 65 - protein, 10 aa, Mus musculus
FFGNLMDASK

SEQ ID NO: 66 - protein, 20 aa, Mus musculus
FKPFTVEIVDSVEAYATMLR

SEQ ID NO: 67 - protein, 16 aa, Mus musculus
FNISNGGPAPEAITDK

SEQ ID NO: 68 - protein, 12 aa, Mus musculus
FYMTEAIQLIVR

SEQ ID NO: 69 - protein, 13 aa, Mus musculus
IDAMHGVVGPYVK

SEQ ID NO: 70 - protein, 8 aa, Mus musculus
LIFADGSR

SEQ ID NO: 71 - protein, 12 aa, Mus musculus
LSGTGSAGATIR

SEQ ID NO: 72 - protein, 9 aa, Mus musculus
NIFDFNALK

SEQ ID NO: 73 - protein, 12 aa, Mus musculus
QEATLVVGGDGR

SEQ ID NO: 74 - protein, 8 aa, Mus musculus
QQFDLENK

SEQ ID NO: 75 - protein, 16 aa, Mus musculus
SGEHDFGAAFDGDGDR

SEQ ID NO: 76 - protein, 22 aa, Mus musculus
SGEHDFGAAFDGDGDRNMILGK

SEQ ID NO: 77 - protein, 16 aa, Mus musculus
SMPTSGALDRVANATK

SEQ ID NO: 78 - protein, 20 aa, Mus musculus
TIEEYAICPDLKVDLGVLGK

SEQ ID NO: 79 - protein, 15 aa, Mus musculus
TQAYPDQKPGTSGLR

SEQ ID NO: 80 - protein, 7 aa, Mus musculus
VSQLQER

SEQ ID NO: 81 - protein, 13 aa, Mus musculus
YDYEEVEAEGANK

SEQ ID NO: 82 - protein, 17 aa, Mus musculus
AAVEEGIVLGGGCALLR

SEQ ID NO: 83 - protein, 8 aa, Mus musculus
APGFGDNR

SEQ ID NO: 84 - protein, 9 aa, Mus musculus
APGFGDNRK

SEQ ID NO: 85 - protein, 6 aa, Mus musculus
FGADAR

SEQ ID NO: 86 - protein, 8 aa, Mus musculus
GANPVEIR

SEQ ID NO: 87 - protein, 12 aa, Mus musculus
GYISPYFINTSK

SEQ ID NO: 88 - protein, 8 aa, Mus musculus
IGIEIIKR

SEQ ID NO: 89 - protein, 19 aa, Mus musculus
IQEITEQLDITTSEYEKEK

SEQ ID NO: 90 - protein, 18 aa, Mus musculus
ISSVQSIVPALEIANAHR

SEQ ID NO: 91 - protein, 22 aa, Mus musculus
KPLVIIAEDVDGEALSTLVLNR

SEQ ID NO: 92 - protein, 11 aa, Mus musculus
LKVGLQVVAVK

SEQ ID NO: 93 - protein, 24 aa, Mus musculus
LVQDVANNTNEEAGDGTTTATVLA

SEQ ID NO: 94 - protein, 16 aa, Mus musculus
TLNDELEIIEGMKFDR

SEQ ID NO: 95 - protein, 9 aa, Mus musculus
VGLQVVAVK

SEQ ID NO: 96 - protein, 9 aa, Mus musculus
VTDALNATR

SEQ ID NO: 97 - protein, 20 aa, Mus musculus
AKPNEVVFLDDFGSNLKPAR

SEQ ID NO: 98 - protein, 19 aa, Mus musculus
ATEIGGILVNTPEDPNLSK

SEQ ID NO: 99 - protein, 20 aa, Mus musculus
AVASLNTPFMPPDPDVSPMK

SEQ ID NO: 100 - protein, 10 aa, Mus musculus
DIVLRPEMSK

SEQ ID NO: 101 - protein, 17 aa, Mus musculus
DMGMVTILVHNTASALR

SEQ ID NO: 102 - protein, 10 aa, Mus musculus
RSEEALALPR

SEQ ID NO: 103 - protein, 9 aa, Mus musculus
SEEALALPR

SEQ ID NO: 104 - protein, 14 aa, Mus musculus
SINRPMLQAAIALK

SEQ ID NO: 105 - protein, 15 aa, Mus musculus
SINRPMLQAAIALKK

SEQ ID NO: 106 - protein, 12 aa, Mus musculus
YQIPALAQAGFR

SEQ ID NO: 107 - protein, 15 aa, Mus musculus
AVFVDLEPTVIDEVR

SEQ ID NO: 108 - protein, 9 aa, Mus musculus
EDAANNYAR

SEQ ID NO: 109 - protein, 9 aa, Mus musculus
EIIDLVLDR

SEQ ID NO: 110 - protein, 21 aa, Mus musculus
FDGALNVDLTEFQTNLVPYPR

SEQ ID NO: 111 - protein, 16 aa, Mus musculus
IHFPLATYAPVISAEK

SEQ ID NO: 112 - protein, 14 aa, Mus musculus
LIGQIVSSITASLR

SEQ ID NO: 113 - protein, 14 aa, Mus musculus
NLDIERPTYTNLNR

SEQ ID NO: 114 - protein, 12 aa, Mus musculus
QLFHPEQLITGK

SEQ ID NO: 115 - protein, 20 aa, Mus musculus
TIGGGDDSFNTFFSETGAGK

SEQ ID NO: 116 - protein, 18 aa, Mus musculus
VGINYQPPTVVPGGDLAK

SEQ ID NO: 117 - protein, 12 aa, Mus musculus
AVMVSNVLLINK

SEQ ID NO: 118 - protein, 10 aa, Mus musculus
EFDALLINPR

SEQ ID NO: 119 - protein, 8 aa, Mus musculus
FLYLGDDR

SEQ ID NO: 120 - protein, 18 aa, Mus musculus
FLYLGDDRNIEEVYVGGK

SEQ ID NO: 121 - protein, 8 aa, Mus musculus
FVSEMLQK

SEQ ID NO: 122 - protein, 20 aa, Mus musculus
IGLGTDVAGGYSYSMLDAIR

SEQ ID NO: 123 - protein, 12 aa, Mus musculus
IVFLEESSQQEK

SEQ ID NO: 124 - protein, 10 aa, Mus musculus
NIEEVYVGGK

SEQ ID NO: 125 - protein, 15 aa, Mus musculus
NYTDVYDKNNLLTNK

SEQ ID NO: 126 - protein, 13 aa, Mus musculus
RAVMVSNVLLINK

SEQ ID NO: 127 - protein, 11 aa, Mus musculus
STDVAEEVYTR

SEQ ID NO: 128 - protein, 21 aa, Mus musculus
THDLYIQSHISENREEIEAVK

SEQ ID NO: 129 - protein, 9 aa, Mus musculus
TPPLALVFR

SEQ ID NO: 130 - protein, 8 aa, Mus musculus
VKPIVTPR

SEQ ID NO: 131 - protein, 8 aa, Mus musculus
YTFPTEQR

SEQ ID NO: 132 - protein, 18 aa, Mus musculus
AAVPSGASTGIYEALELR

SEQ ID NO: 133 - protein, 20 aa, Mus musculus
AGYTDQVVIGMDVAASEFYR

SEQ ID NO: 134 - protein, 19 aa, Mus musculus
DATNVGDEGGFAPNILENK

SEQ ID NO: 135 - protein, 6 aa, Mus musculus
EIFDSR

SEQ ID NO: 136 - protein, 20 aa, Mus musculus
FTASAGIQVVGDDLTVTNPK

SEQ ID NO: 137 - protein, 13 aa, Mus musculus
GNPTVEVDLYTAK

SEQ ID NO: 138 - protein, 11 aa, Mus musculus
GVSQAVEHINK

SEQ ID NO: 139 - protein, 14 aa, Mus musculus
IDKLMIEMDGTENK

SEQ ID NO: 140 - protein, 10 aa, Mus musculus
IGAEVYHNLK

SEQ ID NO: 141 - protein, 17 aa, Mus musculus
LAMQEFMILPVGASSFR

SEQ ID NO: 142 - protein, 8 aa, Mus musculus
SFRNPLAK

SEQ ID NO: 143 - protein, 9 aa, Mus musculus
SGKYDLDFK

SEQ ID NO: 144 - protein, 6 aa, Mus musculus
YDLDFK

SEQ ID NO: 145 - protein, 12 aa, Mus musculus
YITPDQLADLYK

SEQ ID NO: 146 - protein, 6 aa, Mus musculus
YNQILR

SEQ ID NO: 147 - protein, 16 aa, Mus musculus
APFALQVNTLPLNFDK

SEQ ID NO: 148 - protein, 14 aa, Mus musculus
EVLVTIESSGTFSK

SEQ ID NO: 149 - protein, 14 aa, Mus musculus
GSGSGCVYLQTSLK

SEQ ID NO: 150 - protein, 10 aa, Mus musculus
KLQDQPNIQR

SEQ ID NO: 151 - protein, 7 aa, Mus musculus
LLLQEVR

SEQ ID NO: 152 - protein, 11 aa, Mus musculus
LPDLPGNYVTK

SEQ ID NO: 153 - protein, 9 aa, Mus musculus
LQDQPNIQR

SEQ ID NO: 154 - protein, 13 aa, Mus musculus
MVSGFIPMKPSVK

SEQ ID NO: 155 - protein, 8 aa, Mus musculus
NLKPAPIK

SEQ ID NO: 156 - protein, 23 aa, Mus musculus
QQNSHGGFSSTQDTVVALQALSK

SEQ ID NO: 157 - protein, 14 aa, Mus musculus
TEVNTNHVLIYIEK

SEQ ID NO: 158 - protein, 9 aa, Mus musculus
TFHVNSGNR

SEQ ID NO: 159 - protein, 8 aa, Mus musculus
YGAATFTR

SEQ ID NO: 160 - protein, 10 aa, Mus musculus
YNILPVADGK

SEQ ID NO: 161 - protein, 7 aa, Mus musculus
ADAEVLR

SEQ ID NO: 162 - protein, 8 aa, Mus musculus
ADAEVLRK

SEQ ID NO: 163 - protein, 13 aa, Mus musculus
DLVDDLKSELTGK

SEQ ID NO: 164 - protein, 15 aa, Mus musculus
ETSGNLEQLLLAVVK

SEQ ID NO: 165 - protein, 8 aa, Mus musculus
FITIFGTR

SEQ ID NO: 166 - protein, 11 aa, Mus musculus
GAGTDDHTLIR

SEQ ID NO: 167 - protein, 16 aa, Mus musculus
GLGTDEDSILNLLTSR

SEQ ID NO: 168 - protein, 12 aa, Mus musculus
GTVTDFPGFDGR

SEQ ID NO: 169 - protein, 12 aa, Mus musculus
KNFATSLYSMIK

SEQ ID NO: 170 - protein, 10 aa, Mus musculus
LIVAMMKPSR

SEQ ID NO: 171 - protein, 8 aa, Mus musculus
LYDAYELK

SEQ ID NO: 172 - protein, 10 aa, Mus musculus
MLVVLLQANR

SEQ ID NO: 173 - protein, 11 aa, Mus musculus
NFATSLYSMIK

SEQ ID NO: 174 - protein, 8 aa, Mus musculus
QEIAQEFK

SEQ ID NO: 175 - protein, 23 aa, Mus musculus
QVYEEEYGSNLEDDVVGDTSGYY

SEQ ID NO: 176 - protein, 9 aa, Mus musculus
SEIDLFNIR

SEQ ID NO: 177 - protein, 10 aa, Mus musculus
SEIDLFNIRK

SEQ ID NO: 178 - protein, 15 aa, Mus musculus
SIPAYLAETLYYAMK

SEQ ID NO: 179 - protein, 9 aa, Mus musculus
TPEELSAIK

SEQ ID NO: 180 - protein, 9 aa, Mus musculus
VLTEIIASR

SEQ ID NO: 181 - protein, 7 aa, Mus musculus
WGTDEEK

SEQ ID NO: 182 - protein, 15 aa, Mus musculus
YMTISGFQIEETIDR

SEQ ID NO: 183 - protein, 21 aa, Mus musculus
DLYANTVLSGGTTMYPGIADR

SEQ ID NO: 184 - protein, 14 aa, Mus musculus
EKMTQIMFETFNTR

SEQ ID NO: 185 - protein, 10 aa, Mus musculus
GYSFTTTAER

SEQ ID NO: 186 - protein, 7 aa, Mus musculus
IIAPPER

SEQ ID NO: 187 - protein, 8 aa, Mus musculus
IIAPPERK

SEQ ID NO: 188 - protein, 16 aa, Mus musculus
SYELPDGQVITIGNER

SEQ ID NO: 189 - protein, 18 aa, Mus musculus
VAPEEHPVLLTEAPLNPK

SEQ ID NO: 190 - protein, 21 aa, Mus musculus
FLYANTVLSGGSTMFPGIADR

SEQ ID NO: 191 - protein, 7 aa, Mus musculus
IIAPPER

SEQ ID NO: 192 - protein, 8 aa, Mus musculus
IIAPPERK

SEQ ID NO: 193 - protein, 13 aa, Mus musculus
QEYDESGPSIVHR

SEQ ID NO: 194 - protein, 16 aa, Mus musculus
SYELPDGQVITIGNER

SEQ ID NO: 195 - protein, 12 aa, Mus musculus
DFTPVCTTELGR

SEQ ID NO: 196 - protein, 19 aa, Mus musculus
IRFHDFLGDSWGILFSHPR

SEQ ID NO: 197 - protein, 17 aa, Mus musculus
KGESVMVVPTLSEEEAK

SEQ ID NO: 198 - protein, 9 aa, Mus musculus
LPFPIIDDK

SEQ ID NO: 199 - protein, 11 aa, Mus musculus
LPFPIIDDKGR

SEQ ID NO: 200 - protein, 11 aa, Mus musculus
LSILYPATTGR

SEQ ID NO: 201 - protein, 7 aa, Mus musculus
NFDEILR

SEQ ID NO: 202 - protein, 21 aa, Mus musculus
PGGLLLGDEAPNFEANTTIGR

SEQ ID NO: 203 - protein, 9 aa, Mus musculus
VVFIFGPDK

SEQ ID NO: 204 - protein, 10 aa, Mus musculus
VVFIFGPDKK

SEQ ID NO: 205 - protein, 13 aa, Mus musculus
HVFGESDELIGQK

SEQ ID NO: 206 - protein, 14 aa, Mus musculus
RHVFGESDELIGQK

SEQ ID NO: 207 - protein, 13 aa, Mus musculus
TATPQQAQEVHEK

SEQ ID NO: 208 - protein, 16 aa, Mus musculus
VTNGAFTGEISPGMIK

SEQ ID NO: 209 - protein, 9 aa, Mus musculus
ARPALEDLR

SEQ ID NO: 210 - protein, 11 aa, Mus musculus
DFANVYVDAVK

SEQ ID NO: 211 - protein, 6 aa, Mus musculus
ESLAQR

SEQ ID NO: 212 - protein, 6 aa, Mus musculus
LGPLTR

SEQ ID NO: 213 - protein, 7 aa, Mus musculus
LQELQGR

SEQ ID NO: 214 - protein, 9 aa, Mus musculus
LSPVAEEFR

SEQ ID NO: 215 - protein, 11 aa, Mus musculus
LSPVAEEFRDR

SEQ ID NO: 216 - protein, 5 aa, Mus musculus
QEMNK

SEQ ID NO: 217 - protein, 9 aa, Mus musculus
QKLQELQGR

SEQ ID NO: 218 - protein, 11 aa, Mus musculus
SNPTLNEYHTR

SEQ ID NO: 219 - protein, 11 aa, Mus musculus
TQLAPHSEQMR

SEQ ID NO: 220 - protein, 13 aa, Mus musculus
VAPLGAELQESAR

SEQ ID NO: 221 - protein, 13 aa, Mus musculus
VKDFANVYVDAVK

SEQ ID NO: 222 - protein, 10 aa, Mus musculus
VQPYLDEFQK

SEQ ID NO: 223 - protein, 11 aa, Mus musculus
VQPYLDEFQKK

SEQ ID NO: 224 - protein, 8 aa, Mus musculus
FLIDGFPR

SEQ ID NO: 225 - protein, 20 aa, Mus musculus
IQTYLESTKPIIDLYEEMGK

SEQ ID NO: 226 - protein, 13 aa, Mus musculus
IVPVEITISLLKR

SEQ ID NO: 227 - protein, 13 aa, Mus musculus
KNPDSQYGELIEK

SEQ ID NO: 228 - protein, 16 aa, Mus musculus
MKPLVVFVLGGPGAGK

SEQ ID NO: 229 - protein, 13 aa, Mus musculus
REMDQTMAANAQK

SEQ ID NO: 230 - protein, 11 aa, Mus musculus
SVDEVFGEVVK

SEQ ID NO: 231 - protein, 13 aa, Mus musculus
YGYTHLSAGELLR

SEQ ID NO: 232 - protein, 21 aa, Mus musculus
EQAGGDATENFEDVGHSTDAR

SEQ ID NO: 233 - protein, 13 aa, Mus musculus
FLEEHPGGEEVLR

SEQ ID NO: 234 - protein, 12 aa, Mus musculus
TYIIGELHPDDR

SEQ ID NO: 235 - protein, 12 aa, Mus musculus
DSNNLCLHFNPR

SEQ ID NO: 236 - protein, 15 aa, Mus musculus
FNAHGDANTIVCNTK

SEQ ID NO: 237 - protein, 16 aa, Mus musculus
LNMEAINYMAADGDFK

SEQ ID NO: 238 - protein, 16 aa, Mus musculus
LNMEAINYMAADGDFK

SEQ ID NO: 239 - protein, 8 aa, Mus musculus
LPDGHEFK

SEQ ID NO: 240 - protein, 12 aa, Mus musculus
LPDGHEFKFPNR

SEQ ID NO: 241 - protein, 8 aa, Mus musculus
SFVLNLGK

SEQ ID NO: 242 - protein, 10 aa, Mus musculus
VRGEVASDAK

SEQ ID NO: 243 - DNA, 24 bp, Artificial Sequence
  Synthetic
ccataagtcaaatattcagccaag

SEQ ID NO: 244 - DNA, 22 bp, Artificial Sequence
  Synthetic
tatcaggaagtcaaagtgttgg

SEQ ID NO: 245 - DNA, 21 bp, Artificial Sequence
  Synthetic
gcgtggaattcgatgaaatca

SEQ ID NO: 246 - DNA, 20 bp, Artificial Sequence
  Synthetic
cccgccatctagggttatga

SEQ ID NO: 247 - DNA, 20 bp, Artificial Sequence
  Synthetic
aatagacgttcgggcttgca

SEQ ID NO: 248 - DNA, 21 bp, Artificial Sequence
  Synthetic
ggagcattcgtactggccttt

SEQ ID NO: 249 - DNA, 20 bp, Artificial Sequence
  Synthetic
cccctcagatcctccaaaat

SEQ ID NO: 250 - DNA, 20 bp, Artificial Sequence
  Synthetic
aaccctgcttgcagtctatt

SEQ ID NO: 251 - DNA, 19 bp, Artificial Sequence
  Synthetic
tttcctcgcctgcttcttc

SEQ ID NO: 252 - DNA, 20 bp, Artificial Sequence
  Synthetic
ccccgtctctgtattcaacc

SEQ ID NO: 253 - DNA, 23 bp, Artificial Sequence
  Synthetic
gattactgctctggctcctagca

SEQ ID NO: 254 - DNA, 20 bp, Artificial Sequence
  Synthetic
gccaccgatccacacagagt

(19) United States
(12) Patent Application Publication
(10) Pub. No.: US 2011/0065756 A1
(43) Pub. Date: Mar. 17, 2011

(54) METHODS AND COMPOSITIONS FOR TREATMENT OF OBESITY-RELATED DISEASES

(76) Inventors: Bart M. De Taeye, Chicago, IL (US); Douglas E. Vaughan, Chicago, IL (US); Bruce D. Hammock, Davis, CA (US)

(21) Appl. No.: 12/884,317

(22) Filed: Sep. 17, 2010

Related U.S. Application Data
(60) Provisional application No. 61/243,331, filed on Sep. 17, 2009.

(51) Int. Cl. A61K 31/4439; A61K 31/427; A61K 31/4465; A61P 9/00; A61P 7/10; A61P 3/10; A61P 3/04

(52) U.S. Cl. 514/342; 514/369; 514/354

(57) ABSTRACT

The invention relates to methods, compositions, and kits for combination therapy of diabetes and diabetes-related diseases and conditions (e.g., obesity). The invention finds use in patients suffering from or at risk for diabetes or diabetes-related conditions generally, and in patients also suffering from or at risk for fluid retention (e.g., edema, congestive heart failure) and/or in need of synergistic anti-inflammatory, anti-hypertensive, and anti-diabetic effect.

### CROSS-REFERENCE TO RELATED APPLICATION

The present application claims priority to U.S. Provisional application 61/243,331, filed Sep. 17, 2009, which is herein incorporated by reference in its entirety.

### STATEMENT REGARDING FEDERALLY SPONSORED RESEARCH OR DEVELOPMENT

This invention was made with government support under Grant Number SCCOR 5P50HL081009-04001 awarded by the National Institutes of Health and Grant Number 5R37 ES002710 by the National Institute of Environmental Health Sciences. The government has certain rights in the invention.

### FIELD OF THE INVENTION

The invention relates to methods, compositions, and kits for the treatment of diabetes and diabetes-related diseases and conditions (e.g., obesity). Specifically, the invention relates to methods involving combination therapy wherein peroxisome proliferator-activated receptor gamma (PPARγ) is activated and soluble epoxide hydrolase is inhibited. The invention finds use in patients suffering from or at risk for diabetes or diabetes-related conditions, and particularly in patients also suffering from or at risk for fluid retention (e.g., edema, congestive heart failure).

### BACKGROUND OF THE INVENTION

Diabetes mellitus is one of the leading causes of morbidity and mortality in the United States because of its role in the development of optic, renal, neuropathic, and cardiovascular disease. These complications, particularly cardiovascular disease (˜50-75% of medical expenditures), are the major sources of expense and suffering for patients with diabetes mellitus. Approximately two thirds of people with diabetes die from heart disease or stroke. Men with diabetes face a 2-fold increased risk for coronary heart disease, and women have a 3- to 4-fold increased risk. In 1994, 1 of every 7 health care dollars in the United States was spent on patients with diabetes mellitus. The 2002 estimate for direct medical costs due to diabetes in the United States was $92 billion, with another $40 billion in indirect costs. Approximately 20% of Medicare funds are spent on patients with diabetes. Diabetes mellitus is the leading cause of blindness in working-age adults in the United States (e.g., 12,000-24,000 newly blind individuals due to diabetic retinopathy every year); the leading cause of end-stage renal disease (ESRD) in the United States accounting for 44% of all new cases; and the leading cause of nontraumatic lower limb amputation in the United States (e.g., 71,000 amputations occurring in 2004).

One of the newer classes of drugs developed for the treatment of diabetes is thiazolidinediones (also referred to as glitazones or TZDs). TZDs reduce insulin resistance in the periphery (i.e., sensitize muscle and fat to the actions of insulin) and perhaps to a small degree in the liver (i.e., insulin sensitizers, antihyperglycemics). They activate peroxisome proliferator-activated receptor (PPAR) gamma, a nuclear transcription factor that is important in fat cell differentiation and fatty acid metabolism. Their major action is contemplated to be fat redistribution, and they may have beta cell preservation properties. Their glycemic efficacy is moderate, between alpha-glucosidase inhibitors and sulfonylureas.

TZDs require the presence of insulin to work. They generally decrease triglycerides and increase HDL-C, but they increase LDL-C (perhaps large buoyant LDL, which may be less atherogenic). While TZDs have many desirable anti-inflammatory and vascular health effects, edema and weight gain are particularly problematic adverse effects in patients undergoing TZD therapy, especially when TZDs are co-administered with insulin or insulin secretagogues. These effects may induce or worsen congestive heart failure in patients with left ventricular compromise and occasionally in patients with normal left ventricular function. Therefore, these agents are contraindicated in patients with New York Heart Association class III or IV heart failure.

Better pharmacological regimes for the treatment of diabetes are needed. Furthermore, improved methods of diabetes treatment are needed in diabetic patients suffering from or at risk for edema and/or congestive heart failure.

### SUMMARY OF THE INVENTION

Methods of embodiments of the present invention are directed towards combination therapy involving PPARγ-activating agents (e.g., TZDs) in concert with agents inhibiting soluble epoxide hydrolase (sEH). The enzyme sEH hydrolyzes epoxyeicosatrienoic acids (EETs), which are endogenous epoxylipins that have anti-inflammatory, anti-hypertensive, and natriuretic effects, to inactive dihydroxyeicosatrienoic acids (DHETs).

Thiazolidinediones (TZDs), a class of drugs activating the peroxisome proliferator-activated receptor (PPAR) γ, are used to treat type 2 diabetes. While TZDs improve insulin sensitivity and reduce inflammation and hypertension, they lead to fluid retention in part because of sodium retention. Epoxyeicosatrienoic acids (EETs), epoxylipins derived from arachidonic acid by CYP450 epoxygenases, exert an anti-inflammatory and anti-hypertensive effect. The decrease in blood pressure is in part due to the natriuretic effect of EETs. Soluble epoxide hydrolase (sEH) hydrolyzes EETs to inactive dihydroxyeicosatrienoic acids (DHETs). As such, sEH inhibition ameliorates inflammation and hypertension. In addition, sEH inhibitors are appropriate for treatment of type 2 diabetes. TZDs induce the expression of sEH in adipose tissue and more specifically in adipocytes. Based on the functional properties of TZDs and EETs, methods of the present invention are directed towards a combination therapy of TZDs and sEH inhibitors. This combination results in more pronounced anti-hypertensive, anti-inflammatory and anti-diabetic effects, while the increased level of EETs reduces the fluid retention caused by TZDs. In addition, sEH inhibition counteracts the PPARγ-induced sEH activity.

Methods of some embodiments of the present invention are directed towards the use of PPARγ ligands. Such ligands include but are not limited to thiazolidinediones (TZDs, also known as glitazones). Examples of TZDs include but are not limited to rosiglitazone, pioglitazone, and troglitazone. Methods of some embodiments of the present invention are directed towards the use of inhibitors of soluble epoxide hydrolase (sEH). Examples of sEH inhibitors include but are not limited to urea inhibitors of sEH (e.g., DCU, AUDA, APAU, AEPU, TCUB, TPAU, APAU, ACUB, t-ACUB and TUPS); non-urea inhibitors of sEH (e.g., as described in Xie et al. (2009) Bioorgan. Med. Chem. Lett. 19:2354-2359; Anandan et al. (2009) Bioorg. Med. Chem. Lett. 19:1066-1070; each herein incorporated by reference in its entirety) and sEH inhibitors described in U.S. patent application Ser. Nos. 10/328,495; 10/056,284; 10/694,641; 10/817,334; 10/970,373; 10/815,425; 11/189,964; 11/240,444; 11/234,845; 11/330,033; 11/256,685; 11/566,171; 11/685,674; 11/961,881; 12/063,653; 11/995,379; 11/719,092, each herein incorporated by reference its entirety. Additional sEH inhibitors are described in Chiamvimonvat et al. (2007) J. Cardiovasc. Pharmacol. 50:225-237 and Xie et al. (2009 Bioorgan. Med. Chem. Lett. 19:2354-2359; each herein incorporated by reference in its entirety.

In certain embodiments, methods of the present invention find use in the treatment of subjects with diabetes or a diabetes-related condition involving e.g., impaired glucose tolerance, impaired insulin sensitivity, impaired insulin production. Such conditions and disease states include but are not limited to diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus.

In certain embodiments, methods of the present invention find use in the treatment of subjects who are overweight or obese, who may also have or may be at risk of having diabetes or a diabetes-related condition as listed supra, or other conditions such as hypertension or cardiovascular disease. The most widely accepted clinical definition of obesity is the World Health Organization (WHO) criteria based on body mass index (BMI). Under this convention for adults, grade 1 overweight (commonly and simply called overweight) is a BMI of 25-29.9 kg/m². Grade 2 overweight (commonly called obesity) is a BMI of 30-39.9 kg/m². Grade 3 overweight (commonly called severe or morbid obesity) is a BMI greater than or equal to 40 kg/m². In the surgical literature a BMI greater than 40 kg/m² is described as severe obesity, a BMI of 40-50 kg/m² is termed morbid obesity, and a BMI greater than 50 kg/m² is termed super obese. The definition of obesity in children involves BMIs greater than the 85th (commonly used to define overweight) or the 95th (commonly used to define obesity) percentile, respectively, for age-matched and sex-matched control subjects. Secondary causes of obesity include but are not limited to hypothyroidism, Cushing syndrome, insulinoma, hypothalamic obesity, polycystic ovarian syndrome, genetic syndromes (eg, Prader-Willi syndrome, Alström syndrome, Bardet-Biedl syndrome, Cohen syndrome, Börjeson-Forssman-Lehmann syndrome, Fröhlich syndrome), growth hormone deficiency, oral contraceptive use, medication-induced obesity (eg, phenothiazines, sodium valproate, carbamazepine, tricyclic antidepressants, lithium, glucocorticoids, megestrol acetate, thiazolidine diones, sulphonylureas, insulin, adrenergic antagonists, serotonin antagonists [especially cyproheptadine]), eating disorders (especially binge-eating disorder, bulimia nervosa, night-eating disorder), hypogonadism, pseudohypoparathyroidism, and obesity related to tube feeding.

In certain embodiments, methods of the present invention find use in the treatment of congestive heart failure (CHF), the severe manifestation of which is pulmonary edema. CHF is an imbalance in pump function in which the heart fails to adequately maintain the circulation of blood. CHF can be categorized as forward or backward ventricular failure. Backward failure is secondary to elevated systemic venous pressure, whereas left ventricular failure is secondary to reduced forward flow into the aorta and systemic circulation. Furthermore, heart failure can be subdivided into systolic and diastolic dysfunction. Systolic dysfunction is characterized by a dilated left ventricle with impaired contractility, whereas diastolic dysfunction occurs in a normal or intact left ventricle with impaired ability to relax and receive as well as eject blood. In the New York Heart Association's functional classification of CHF, Class I describes a patient whose normal physical activity is not limited by symptoms. Class II occurs when ordinary physical activity results in fatigue, dyspnea, or other symptoms. Class III is characterized by a marked limitation in normal physical activity. Class IV is defined by symptoms at rest or with any physical activity. CHF and/or pulmonary edema may be caused by coronary artery disease (e.g., secondary to loss of left ventricular muscle), ongoing ischemia, decreased diastolic ventricular compliance, hypertension, valvular heart disease, congenital heart disease, other cardiomyopathies, myocarditis, infectious endocarditis, pregnancy, and hyperthyroidism. CHF is often precipitated by cardiac ischemia or dysrhythmias, cardiac or extracardiac infection, pulmonary embolus, physical or environmental stresses, changes or noncompliance with medical therapy, dietary indiscretion, or iatrogenic volume overload.

In certain embodiments, methods of the present invention find use in the treatment of edema, which is an abnormal accumulation of fluid beneath the skin, or in one or more cavities of the body. Causes of edema which are generalized to the whole body can cause edema in multiple organs and peripherally. For example, severe heart failure (CHF, as described supra) can cause pulmonary edema, pleural effusions, ascites and peripheral edema, the last of which effects can also derive from less serious causes. Organ-specific edema types include cerebral edema, pulmonary edema, pleural efflusions, corneal edema, periorbital edema, cutaneous edema, myxedema, edema of the feet, and lymphedema.

In certain embodiments, methods of the present invention find use in treatment of conditions or physiological states including but not limited to fluid retention, sodium retention, hypertension (high blood pressure), and low levels of epoxyeicosatrienoic acids (EETs).

In some embodiments, methods of the present invention comprise testing a subject for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus, followed by administering a combination therapy comprising a PPARγ agonist and an inhibitor of soluble epoxide hydrolase. In some embodiments, methods of the present invention comprise administering to a subject a combination therapy comprising a PPARγ agonist and an inhibitor of soluble epoxide hydrolase, followed by testing the subject for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus. In some embodiments, methods of the present invention comprise testing a subject for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus, followed by administering a combination therapy comprising a PPARγ agonist and an inhibitor of soluble epoxide hydrolase, followed by a second round of testing for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus. In some embodiments, methods of the present invention comprise testing a subject for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus, followed by administering a combination therapy comprising a PPARγ agonist and an inhibitor of soluble epoxide hydrolase, followed by a second round of testing for diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus, and a second administration of a combination therapy comprising a PPARγ agonist and an inhibitor of soluble epoxide hydrolase, with this second administration being modified in dose, duration, frequency, or administration route in a manner dependent upon the results of the prior testing.

In some embodiments, the invention comprises use of a combination therapy comprising a PPARγ agonist and an inhibitor of soluble epoxide hydrolase in the manufacture of a medicament for the treatment of a condition such as diabetes or a diabetes-related disease or condition such as impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus.

Methods of the present invention are not limited by dosing, formulation, administration routes, or temporal administration regimes of the combination therapy comprising a PPARγ agonist and an inhibitor of soluble epoxide hydrolase. In some embodiments, dosage of the PPARγ agonist is in the range of about 2 mg up to about 2,000 mg per day, with variations necessarily occurring depending on the disease target, the patient, and the route of administration. In some embodiments, dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day. In some embodiments, dosage of the sEH inhibitor is in the range of about 2 mg up to about 2,000 mg per day, with variations necessarily occurring depending on the disease target, the patient, and the route of administration. In some embodiments, dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day. Administration routes include but are not limited to oral, topical, and parenteral (e.g. intravenous, intramuscular, intraperitoneal, subcutaneous, intrathecal, intraarterial, nasal, or pulmonary administration). The invention is not limited by temporal aspects of administration of a PPARγ agonist and a sEH inhibitor. The PPARγ agonist and sEH inhibitor may be administered simultaneously or sequentially without regard to order of administration. The combination therapy may be administered between 1 and 50 times per day, or less often (e.g., every other day, every three days, every 4 days, every 5 days, every 6 days, once a week, once a month, once every three months.) The combination therapy may be continued for 1 day, 1 week, 1 month, 1 year, 10 years, or more than 10 years.

In certain embodiments, the present invention provides a method for treating a disease related to diabetes in a subject comprising administration of a peroxisome proliferator-activated receptor gamma agonist and an inhibitor of soluble epoxide hydrolase. In certain embodiments, the present invention provides a method for preventing edema in a subject with a diabetes-related condition comprising administration of a peroxisome proliferator-activated receptor gamma agonist and an inhibitor of soluble epoxide hydrolase. In some embodiments, the peroxisome proliferator-activated receptor gamma agonist is an agent such as rosiglitazone, pioglitazone, and troglitazone. In some embodiments, the dose of the rosiglitazone is 0.1-100 mg per day (e.g., 0.1-1, 1-2, 2-5, 5-10, 10-20, 20-30, 30-40, 40-50, 50-75, 75-100 mg per day) (e.g., 1-20 mg per day). In some embodiments, the dose of the rosiglitazone is 0.1-100 mg per day (e.g., 0.1-1, 1-2, 2-5, 5-10, 10-20, 20-30, 30-40, 40-50, 50-75, 75-100 mg per day) (e.g., 5-20 mg/kg/day). In some embodiments, the dose of pioglitazone is 0.1-100 mg per day (e.g., 0.1-1, 1-2, 2-5, 5-10, 10-20, 20-30, 30-40, 40-50, 50-75, 75-100 mg per day) (e.g., 5-50 mg per day). In some embodiments, the inhibitor of soluble hydroxide hydrolase is an agent such as 1,3-disubstituted ureas and sulfonyl isonipectotamides. In some embodiments, the 1,3-disubstituted ureas are agents such as APAU, AEPU, AUDA, TCUB, TPAU, TUPS, t-ACUB and ACUB. In some embodiments, the dose of inhibitor of soluble hydroxide hydrolase is 1-150 mg/kg. In some embodiments, the peroxisome proliferator-activated receptor gamma agonist and the inhibitor of soluble epoxide hydrolase are administered in pharmaceutically acceptable formulations. In some embodiments, the peroxisome proliferator-activated receptor gamma agonist and the inhibitor of soluble epoxide hydrolase are administered simultaneously. In some embodiments, the peroxisome proliferator-activated receptor gamma agonist and the inhibitor of soluble epoxide hydrolase are formulated for simultaneous administration in a single delivery agent. In some embodiments, the single delivery agent is formulated for oral administration. In some embodiments, the peroxisome proliferator-activated receptor gamma agonist and the inhibitor of soluble epoxide hydrolase are administered sequentially. In some embodiments, the administration occurs between 1 and 10 times per day. In some embodiments, the administration occurs by routes such as oral or parenteral. In some embodiments, the disease related to diabetes is a disease such as diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus. In some embodiments, the subject is human. In some embodiments, the subject suffers from edema. In some embodiments, the subject is at risk for edema. In some embodiments, the subject suffers from congestive heart failure. In some embodiments, the subject is at risk for congestive heart failure.

In certain embodiments, the present invention provides a method for increasing levels of epoxyeicosatrienoic acids in a diabetic subject comprising administration of a peroxisome proliferator-activated receptor gamma agonist and an inhibitor of soluble epoxide hydrolase. In some embodiments, the levels of epoxyeicosatrienoic acids are elevated 1.5-fold or higher relative to levels present prior to the administration of the peroxisome proliferator-activated receptor gamma agonist and the inhibitor of soluble epoxide hydrolase.

In certain embodiments, the present invention provides a method for treating congestive heart failure in a diabetic subject comprising administration of a peroxisome proliferator-activated receptor gamma agonist and an inhibitor of soluble epoxide hydrolase. In some embodiments, the subject has congestive heart failure. In some embodiments, the subject is at risk of congestive heart failure.

In certain embodiments, the present invention provides a method for preventing edema in a patient undergoing PPAR gamma agonist therapy comprising administering an inhibitor of soluble epoxide hydrolase.

In certain embodiments, the present invention provides a method of promoting natriuresis in a subject with diabetes comprising administration of a peroxisome proliferator-activated receptor gamma agonist and an inhibitor of soluble epoxide hydrolase.

In certain embodiments, the present invention provides a composition comprising a first amount of a peroxisome proliferator-activated receptor gamma agonist and a second amount of an inhibitor of soluble epoxide hydrolase, wherein the first amount and second amount together comprise a therapeutically effective amount of the peroxisome proliferator-activated receptor gamma agonist and the inhibitor of soluble epoxide hydrolase for use as a medicament. In some embodiments, the invention provides a pharmaceutical preparation comprising a composition comprising a first amount of a peroxisome proliferator-activated receptor gamma agonist and a second amount of an inhibitor of soluble epoxide hydrolase, wherein the first amount and second amount together comprise a therapeutically effective amount of the peroxisome proliferator-activated receptor gamma agonist and the inhibitor of soluble epoxide hydrolase for use as a medicament.

Additional embodiments will be apparent to persons skilled in the relevant art based on the teachings contained herein.

### DESCRIPTION OF THE DRAWINGS

FIG. 1 shows weight gain for C57BL/6J animals used for proteomic analysis. Animals fed a diet high in fat (HFD) gained significantly more weight (a, p=0.004) than animals fed a ‘standard fat diet’ (SFD). *p<0.05; **p<0.01 vs SFD. n=5.

FIG. 2 shows sEH mRNA levels determined by RT-QPCR. All data are expressed relative to liver SFD. *p<0.001 vs liver for the same diet; **p<0.01 vs kidney for the same diet. Diet had no effect on sEH mRNA levels (p>0.05 for each organ). n=5.

FIG. 3 shows sEH activity for liver, kidney and epididymal fat pad, expressed per mg protein (a) and for the total organ after normalization for body weight (b). *p<0.01 vs SFD. n=5.

FIG. 4 shows expression of sEH in adipose tissue. Kidney was used as a positive control. While most tubuli stained positive (a), glomeruli (arrows) lacked sEH as expected. In adipose tissue from lean (b, d) and obese (c) mice sEH expression was detected in endothelial cells (b, d), macrophages (c) and adipocytes (b, c). Endothelial cells were identified based on their presence in the blood vessel, while adipocytes and macrophages, organized in a typical crown-like structure, were distinguished based on their morphology. Adipose (e) and kidney (f) sections with omission of the anti-sEH IgG fraction did not show any detection. Bars for kidney and adipose sections are 200 and 50 μm, respectively.

FIG. 5 shows sEH in 3T3-L1 cells. Differentiation of 3T3-L1 preadipocytes was analyzed by oil red O staining at days 0, 2, 4 and 7 (left to right) (a). Bars, 50 μm. Western blotting for perilipin A and sEH indicated that sEH protein levels increased during preadipocyte differentiation (b), densitometric analysis (rdu; relative densitometric units); (c) representative bands for perilipin A, perilipin B, sEH and control actin). n=3. aP2, sEH and Pref-1 mRNA levels determined by RT-QPCR indicated that sEH mRNA levels increased during preadipocyte differentiation (d). n=6.

FIG. 6 shows EET (a, c) and DHET (b,d) levels in 3T3-L1 cells (a, b) and cell culture medium (c, d) during adipogenesis. EET and DHET levels are generally higher in the 3T3-L1 preadipocytes (day 0) than after 4 and 8 days of induction of differentiation. Levels at day 4 and 8 for some samples were below the level of detection (LD) (no statistical analysis possible). †p<0.05; ‡p<0.01; *p<0.001 vs day 0. n=3.

FIG. 7 shows that EET producing CYP450 enzymes are expressed in adipose tissue and 3T3-L1 adipocytes. Using reverse transcriptase reactions for some different murine CYP450s known to produce EETs, it was shown that CYP2C55 and CYP2J9 were expressed in epididymal fat (F); liver (L) was used as a positive control (a). CYP2C primers recognize the different CYP2Cs and thus account for additional CYP2Cs in addition to those that were individually tested. CYP2C55 was not amplified by these primers potentially due to a lower reaction efficiency. CYP2J9 and CYP2C55 were expressed both in epididymal fat of mice fed regular chow (SF) and the ‘western diet’ (HF) (b). Finally, also in 3T3-L1 adipocytes CYP2C55 and CYP2J9 were expressed (c). The same DNA marker was used for a, b and c.

FIG. 8 shows that troglitazone regulates sEH expression in mature 3T3-L1 adipocytes. (a, b) sEH mRNA levels in 3T3-L1 adipocytes determined by RT-QPCR during incubation with troglitazone (a, 10 μmol/L; b, 48 hrs). n=6. (c, d, e, f) Western blotting of sEH and control actin in 3T3-L1 adipocytes during incubation with troglitazone (c, e, 10 μmol/L; d, f, 48 hrs) (rdu; relative densitometric units). (e, f) Western blots used for quantitation in c and d, respectively. n=3. †p<0.05; *p<0.01;**p<0.001 vs baseline.

FIG. 9 shows characterization of C57BL/6J animals used for treatment with rosiglitazone. While gavaging of mice generally resulted in weight loss, rosiglitazone itself did not influence weight (a). Rosiglitazone treatment reduced plasma glucose levels (b) and plasma insulin levels (c) determined after a 6 hour fasting period. †p<0.05 vs before treatment; *p<0.05 vs vehicle. n=5.

FIG. 10 shows results of an intraperitoneal glucose tolerance test (IPGTT) after rosiglitazone vs vehicle treatment. While plasma glucose levels were lower after a 16 hour fasting period in the rosiglitazone treated animals (b), no difference was observed in overall plasma glucose levels during the IPGTT using two-way ANOVA (a). Rosiglitazone reduced plasma insulin levels after a 16 hour fast (d), while ANOVA did not reveal a difference for the insulin levels during the IPGTT (c). While the power of the study (n=5) is too low to observe a significant difference for a and c, b and d clearly show that rosiglitazone exerted the expected effect. *p<0.05 vs vehicle. n=5.

FIG. 11 shows that rosiglitazone increases sEH expression in adipose tissue. sEH mRNA levels (a) and activity (b) were increased in the epididymal fat pad of obese C57BL/6J mice by rosiglitazone. Rosiglitazone also increased sEH expression in subcutaneous fat whereas levels of kidney and liver sEH mRNA were not influenced (c). n=5. *p<0.05 vs vehicle;**p<0.05 vs epididymal fat.

FIG. 12 shows Expression of sEH in epididymal adipose tissue of mice treated with rosiglitazone (a-c) vs vehicle (d-f). Adipose tissue sections with omission of the anti-sEH IgG fraction did not show any detection (g-i).

FIGS. 13 a-f show DIGE analysis using DeCyder 2D v6.5. MS acquisition/processing: 4000 Explorer Series v3.0 (2000 laser shots per PMF, 2000 per ms/ms). S/N<3 peak filtering, local noise window width=250, min. peak width at FWHM=2.9, without baseline correction or smoothing. Database searching: GPS Explorer v3.6 running the MASCOT v1.9 suite of database search algorithms. Searches performed at 200 ppm peptide mass accuracy without constraining database: Sprot20070410 (283454 sequences; 104030551 residues).

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having diabetes” refers to a subject that presents one or more symptoms indicative of diabetes or a diabetes-related condition (e.g., diabetes mellitus type 1, diabetes mellitus type 2, gestational diabetes, pre-diabetes, metabolic syndrome, syndrome X) (e.g., polyuria, polydipsia, nocturia, fatigue, weight loss) or is being screened for diabetes (e.g., during a routine physical). A subject suspected of having diabetes or a diabetes-related condition may also have one or more risk factors. A subject suspected of having diabetes or a diabetes-related condition has generally not been tested for diabetes or a diabetes-related condition. However, a “subject suspected of having diabetes” encompasses an individual who has received a preliminary diagnosis (e.g., a random plasma glucose level) but for whom a confirmatory test (e.g., fasting glucose plasma level) has not been done or for whom the type of diabetes is not known. A “subject suspected of having diabetes” is sometimes diagnosed with diabetes and is sometimes found to not have diabetes.

As used herein, the term “subject diagnosed with diabetes” refers to a subject who has been tested and found to have diabetes or a diabetes-related condition (e.g., diabetes mellitus type 1, diabetes mellitus type 2, gestational diabetes, pre-diabetes, metabolic syndrome, syndrome X) (e.g., a random plasma glucose level of ≧200 mg/dL or greater, a fasting glucose plasma level of ≧126 mg/dL occurring on two separate occasions, 2 hours post glucose load (75 g) plasma glucose of ≧200 mg/dL on two separate occasions). Diabetes may be diagnosed using any suitable method, including but not limited to, measurements of random plasma glucose level, fasting plasma glucose level, hemoglobin A1c (HbA1c or A1c) levels, glycosylated hemoglobin (GHb) levels. A “preliminary diagnosis” is one based only on presenting symptoms (e.g., polyuria, polydipsia, nocturia, fatigue, weight loss).

As used herein, the term “initial diagnosis” refers to a test result of initial diabetes diagnosis that reveals elevated plasma glucose levels (e.g., random plasma glucose levels).

As used herein, the term “subject at risk for diabetes” refers to a subject with one or more risk factors for developing diabetes or a diabetes-related condition. Risk factors include, but are not limited to, obesity (particularly central or abdominal obesity), race, gender, age, genetic predisposition, diet, lifestyle (particularly sedentary lifestyle), and diseases or conditions that can lead to secondary diabetes (e.g., treatment with glucocorticoids, Cushing syndrome, acromegaly, pheochromocytoma).

As used herein, the term “characterizing diabetes in subject” refers to the identification of one or more properties of diabetes disease or a diabetes-related disease in a subject, including but not limited to, plasma glucose levels (random, fasting, or upon glucose challenge); Hemoglobin A1c (HbA1c or A1c) levels; glycosylated hemoglobin (GHb) levels; microalbumin levels or albumin-to-creatinine ratio; insulin levels; C-peptide levels; antibodies to insulin, islet cells, or glutamic acid decarboxylase (GAD); levels of anti-GAD65 antibody (e.g., as an indicator of latent autoimmune diabetes of adults).

As used herein, the term “providing a prognosis” refers to providing information regarding the impact of the presence of diabetes on a subject's future health (e.g., expected morbidity or mortality, the likelihood of getting diabetes, and the risk of diabetes-related complications).

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., diabetes). Test compounds comprise both known and potential therapeutic compounds. A test compound can be determined to be therapeutic by screening using screening methods known in the art.

As used herein, the term “sample” is used in its broadest sense. In one sense, it is meant to include a specimen or culture obtained from any source, as well as any biological. Biological samples may be obtained from animals (including humans) and encompass fluids, solids, tissues, and gases. Biological samples include blood products, such as plasma, serum and the like. Such examples are not however to be construed as limiting the sample types applicable to the present invention.

As used herein, the term “effective amount” refers to the amount of a compound (e.g., a PPARγ-activating compound and an sEH-inhibiting compound as described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., a PPARγ-activating compound and an sEH-inhibiting compound as described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vitro, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. (1975).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “instructions for administering said compound to a subject,” and grammatical equivalents thereof, includes instructions for using the compositions contained in a kit for the treatment of diabetes or diabetes-related conditions (e.g., providing dosing, route of administration, decision trees for treating physicians for correlating patient-specific characteristics with therapeutic courses of action). The PPARγ-activating and/or sEH-inhibiting compounds of the present invention (e.g. as presented herein) can be packaged into a kit, which may include instructions for administering the compounds to a subject.

As used herein, the term “obesity” refers to the state of being in excess of one's ideal weight, wherein determination of ideal weight takes into account multiple factors including but not limited to one's height, age, sex, and build. In some embodiments, “obese” is defined as having a body mass index (BMI) of 30 or greater.

As used herein, the term “body mass index” or “BMI” is defined as a person's weight in kilograms (kg) divided by their height in meters (m) squared. While BMI strongly correlates with overall fat content, one of ordinary skill in the art understands that some very muscular people may have a high BMI without undue health risks.

As used herein, the term “diabetes” or “diabetes-related condition” refers to a metabolic or endocrinological disease, condition, or state affecting control of glycemia. Diabetes or diabetes-related conditions may affect sensitivity to insulin or production of insulin. Examples of diabetes or diabetes-related diseases or condition include but are not limited to impaired insulin signaling, diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus.

As used herein, the term “edema” refers to an abnormal accumulation of fluid beneath the skin, or in one or more cavities of the body. Causes of edema which are generalized to the whole body can cause edema in multiple organs and peripherally. For example, severe heart failure (CHF, as described supra) can cause pulmonary edema, pleural effusions, ascites and peripheral edema, the last of which effects can also derive from less serious causes. Organ-specific edema types include cerebral edema, pulmonary edema, pleural efflusions, corneal edema, periorbital edema, cutaneous edema, myxedema, edema of the feet, and lymphedema.

As used herein, the term “congestive heart failure” refers to an imbalance in pump function in which the heart fails to adequately maintain the circulation of blood. In the New York Heart Association's functional classification of CHF, Class I describes a patient whose normal physical activity is not limited by symptoms. Class II occurs when ordinary physical activity results in fatigue, dyspnea, or other symptoms. Class III is characterized by a marked limitation in normal physical activity. Class IV is defined by symptoms at rest or with any physical activity.

As used herein, the term “a thiazolidinedione” or “a TZD” (also known as a member of the glitazones family) refers to a compound that is structurally related to thiazolidinedione. Examples of thiazolidiones include but are not limited to rosiglitazone, pioglitazone, and troglitazone.

As used herein, the term “PPARγ agonist” refers to an agent that increases the activity of PPARγ by at least 0.5-fold.

As used herein, the term “soluble epoxide hydrolase inhibitor” refers to an agent that inhibits the activity of soluble epoxide hydrolase enzyme by at least 20%.

### DETAILED DESCRIPTION OF THE INVENTION

Thiazolidinediones (TZDs) are agonists of the peroxisome proliferator-activated receptor (PPAR) γ nuclear receptor. PPARγ regulates glucose and fat metabolism, adipogenesis, inflammation, and plays a role in a variety of disorders such as atherosclerosis and cancer (reviewed in Tontonoz et al. (2008) 77:-289-312; herein incorporated by reference in its entirety). TZDs can be prescribed as insulin-sensitizing drugs to treat type 2 diabetes mellitus (Yki-Jarvinen et al. (2004) New Engl. J. Med. 351-1106-1118; herein incorporated by reference in its entirety). In spite of their benefits, TZDs have been associated with peripheral edema and worsening of heart failure (Patel et al. (2005) Diab. Vasc. Dis. Res. 2:61-66; herein incorporated by reference in its entirety). Ten to 15% of patients treated with TZDs present with edema, often requiring discontinuation of the therapy (Mudaliar et al. (2003) Endocr. Pract. 9:406-416; Page et al. (2003) Pharmacotherapy 23:945-954; each herein incorporated by reference in its entirety). The occurrence of the edema means that TZDs are contraindicated in patients with New York Heart Association class III and IV congestive heart failure (Nesto et al. (2004) Diabetes Care 27:256-263; each herein incorporated by reference in its entirety). While the use of TZDs has increased concomitantly with the epidemic of type 2 diabetes, so has also the number of diabetic patients with congestive heart failure (King et al. (2004) Am. J. Health-Syst. Pharm. 61:390-393; herein incorporated by reference in its entirety). While the present invention is not limited to any particular mechanism, and an understanding of the mechanism is not necessary to practice the present invention, it is contemplated that a role for PPARγ in renal sodium reabsorption exists and that PPARγ may contribute to TZD-induced edema (Staels et al. (2005) Cell Metab. 2:77-78; herein incorporated by reference in its entirety). While the present invention is not limited to any particular mechanism, and an understanding of the mechanism is not necessary to practice the present invention, it is contemplated that increased Na⁺ absorption through the epithelial Na⁺ channel ENaC and Na—K-ATPase system in the collecting ducts and distal nephron of the kidney are involved, although contradictory findings have also been reported (Vallon et al. (2009) J. Am. Soc. Nephrol. 20:721-729; Chen et al. (2005) J. Pharmacol. Exp. Ther. 312:718-725; Guan et al. (2005) Nat. Med. 11:861-866; Zhang et al. (2005) PNAS 102:9406-9411; each herein incorporated by reference in its entirety).

In the past decade sEH has been of interest with regard to hypertension (Sinal et al. (2000) J. Biol. Chem. 275:40504-10510; Jung et al. (2005) Hypertension 45:759-765; Imig et al. (2002) 39:690-694; Imig et al. (2005) Hypertension 46:975-981; each herein incorporated by reference in its entirety) and inflammation (Node et al. (1999) Science 285:1276-1279; Kozak et al. (2000) Am. J. Physiol. Regul. Integr. Comp. Physiol. 279:R455-4601; Ralck et al. (2003) Bioorg. Med. Chem. Lett. 13:4011-4014; Schmelzer et al. (2005) PNAS 102:9772-9777; Liu et al. (2005) PNAS 102:16747-16752; each herein incorporated by reference in its entirety). In addition, sEH has been associated with atherosclerosis (Ulu et al. (2008) Cardiovasc. Pharmacol. 52:341-323; herein incorporated by reference in its entirety), fatty acid (Newman et al. (2003) PNAS 100:1558-1563; herein incorporated by reference in its entirety) and cholesterol metabolism (EnayetAllah et al. (2008) J. Biol. Chem. 283:36592-36598; herein incorporated by reference in its entirety). While the present invention is not limited to any particular mechanism, and an understanding of the mechanism is not necessary to practice the present invention, it is contemplated that sEH plays a role in the development of insulin resistance (Ohtoshi et al. (2005) Biochem. Biophys. Res. Commun. 331:347-350; herein incorporated by reference in its entirety). While the present invention is not limited to any particular mechanism, and an understanding of the mechanism is not necessary to practice the present invention, it is contemplated that sEH hydrolyzes EETs, generally considered to be an inactivation reaction. EETs have an anti-inflammatory effect, in part because of their function as PPARγ ligands (Liu et al. (2005) PNAS 102:16747-16752; herein incorporated by reference in its entirety), and lead to decreased blood pressure in part as a result of EET-induced vasodilation and inhibition of distal Na⁺ reabsorption (reviewed in Spector et al. (2007) J. Physiol. Cell Physiol. 292:C996-1012; Capdevila et al. (2007) Kidney Intl. 72:683-689; each herein incorporated by reference in its entirety).

Experiments conducted during the course of developing certain embodiments of the present invention found that TZDs can increase mRNA and protein levels and activity of sEH in adipose tissue and more specifically in adipocytes. While the present invention is not limited to any particular mechanism, and an understanding of the mechanism is not necessary to practice the present invention, it is contemplated that the beneficial effects of PPARγ agonists are not fully achieved because of elevated sEH activity, especially in overweight and obese patients. Since members of the latter group are candidates for TZD therapy, improving TZD actions and reducing the hazardous fluid retention associated with TZD treatment is of benefit.

Methods and compositions of some embodiments of the present invention comprise a combination therapy of a TZD with a sEH inhibitor. This combination reduces the Na⁺ retention and associated edema. In addition, while the present invention is not limited to any particular mechanism, and an understanding of the mechanism is not necessary to practice the present invention, it is contemplated that a synergistic effect is achieved with regard to glycemic control, aside from that for blood pressure regulation and inflammation. Additionally, combination therapy makes TZDs available as a treatment option for patients with congestive heart failure.

Experiments conducted during the course of developing some embodiments of the present invention (e.g., Example 1) show that sEH is expressed and synthesized in 3T3-L1 cells and adipose tissue. Although the expression of sEH has been identified in numerous tissues, including the liver and kidney (Enayetallah et al. (2004) J. Histochem. Cytochem. 52:447-454; Sinal et al. (2000) J. Biol. Chem. 275:40504-40510; each herein incorporated by reference in its entirety), there are no previous reports describing adipose tissue, or adipocytes more specifically, as a source of sEH. In addition, thiazolidinediones, a class of PPARγ agonists that stimulate adipogenesis/lipogenesis as described supra (Tamori et al. (2002) Diabetes 51:2045-2055; Hamm et al. (1999) Ann. NY Acad. Sci. 892:134-145; each herein incorporated by reference in its entirety), increase expression levels of sEH in 3T3-L1 adipocytes in vitro and adipose tissue in vivo.

Experiments conducted during the course of developing some embodiments of the present invention identify sEH as a contributor to inflammatory amplification in obesity, hypertension, and type 2 diabetes. Furthermore, experiments conducted during the course of developing some embodiments of the present invention identified novel therapeutic candidates contributing to the cardiovascular consequences of obesity. Because sEH contributes to angiotensin II-induced hypertension (Jung et al. (2005) Hypertension 45:759-765; herein incorporated by reference in its entirety), left ventricular hypertrophy (Ai et al. (2009) PNAS 106:564-569; herein incorporated by reference in its entirety), and atherosclerosis (Ulu et al. (2008) J. Cardiovasc. Pharmacol. 52:314-323; herein incorporated by reference in its entirety), its increased production in adipocytes and visceral fat is important. By deactivating EETs, sEH augments inflammation (Schmelzer et al. (2005) PNAS 102:9772-9777; Liu et al. (2005) PNAS 102:1647-1752; each herein incorporated by reference in its entirety), which is known to contribute to local and systemic insulin resistance. While the present invention is not limited to any particular mechanism, and an understanding of the mechanism is not necessary to practice the present invention, it is contemplated that by enhancing the bioavailability of EETs in obesity, sEH inhibition provides a novel approach of reducing inflammation.

Evidence exists regarding a relationship between sEH expression and hypertension (Sinal et al. (2000) 275:40504-40510; Jung et al. (2005) Hypertension 45:759-765; Imig et al. (20020 Hypertension 39:690-694; Imig et al. (2005) Hypertension 46:975-981; Yu et al. (2000) Circ. Res. 87:992-998’ Rodriguez et al. (2006) Ann. NY Acad. Sci. 1079:130-134; Fleming et al. (2007) Trends Pharmacol. Sci. 28:448-452; each herein incorporated by reference in its entirety). However, it remains unknown whether sEH derived from adipocytes influences vascular resistance locally or systemically. Adipose tissue comprises a substantial proportion of total body weight and an extensive capillary network surrounds adipose tissue (Crandall et al. (1997) Microcirculation 4:211-232; herein incorporated by reference in its entirety). EETs cause vasodilation by acting as autocrine or paracrine hormones (Spector et al. (2004) Prog. Lipid Res. 43:55-90; herein incorporated by reference in its entirety). While the present invention is not limited to any particular mechanism, and an understanding of the mechanism is not necessary to practice the present invention, it is contemplated that increased levels of sEH in adipose tissue lead to a decreased release of endogenous EETs and/or an increased clearance of exogenous EETs produced by other cell types in adipose tissue such as endothelial cells. Alternatively, the stimuli that promote sEH expression in fat may also induce increased sEH activity in vascular tissue remote from visceral fat.

While the present invention is not limited to any particular mechanism, and an understanding of the mechanism is not necessary to practice the present invention, it is contemplated that sEH plays a role in adipogenesis. Loss-of-function studies demonstrated that PPARγ plays a critical role in adipogenesis in vivo and in vitro (Kubota et al. (1999) Mol. Cell. 4:597-609; Rosen et al. (1999) Mol. Cell. 4:611-617; each herein incorporated by reference in its entirety). Ligand-activated PPARγ regulates many genes involved in glucose and lipid homeostasis and is therefore involved in the maintenance of normal insulin responsiveness (Walczak et al. (2002) J. Lipid Res. 43:177-186; herein incorporated by reference in its entirety). Prior to experiments conducted during the course of developing some embodiments of the present invention, one of the questions remaining with regard to PPARγ and adipogenesis was the identity of a possible endogenous ligand (Tontonoz et al. (2008) Ann. Rev. Biochem. 77:289-312; herein incorporated by reference in its entirety). Numerous reports have described a wide variety of structurally different molecules that the PPARγ ligand binding domain can accommodate in its large hydrophobic pocket (Nolte et al. (1998) Nature 395:137-143; herein incorporated by reference in its entirety). PPARγ ligands include the thiazolidinediones (Murphy et al. (2000) Trends Pharmacol. Sci. 21:469-474; herein incorporated by reference in its entirety), polyunsaturated fatty acids (Hertzel et al. (1998) Mol. Cell. Biochem. 188:33-39; herein incorporated by reference in its entirety), and arachidonic acid metabolites such as 15-deoxy-Δ^{12,14} prostaglandin J (Forman et al. (1995) Cell 83:803-812; Kliewer et al. (1995) Cell 83:813-819; each herein incorporated by reference in its entirety), among others. Other metabolites derived from arachidonic acid by either the lipoxygenase or cyclooxygenase have been shown to positively or negatively influence adipogenesis (Xie et al. (2006) Diabetes Obes. Metab. 8:83-93; Yan et al. (2003) J. Lipid Res. 44:424-429; each herein incorporated by reference in its entirety).

With regard to the third pathway of arachidonic acid metabolism, the CYP450 epoxygenase pathway, only a limited number of studies have been performed. Madsen et al. reported that inhibition of the lipoxygenase, but not the cyclooxygenase or CYP450 epoxygenase pathway, prevents adipocyte differentiation in 3T3-L1 cells (Madsen et al. (2003) Biochem. J. 375:539-549; herein incorporated by reference in its entirety). However, Liu et al. showed that EETs, generated from arachidonic acid by CYP450 epoxygenases, can bind and activate PPARγ(Liu et al. (2005) PNAS 102:16747-16752; herein incorporated by reference in its entirety). In addition, EETs combined with a sEH inhibitor can induce the expression of aP2 in 3T3-L1 preadipocytes, indicative of adipocyte differentiation (Liu et al. (2005) PNAS 102:16747-16752; herein incorporated by reference in its entirety). Based on this prior observation and in combination with data showing that EETs are present in preadipocytes and that sEH is upregulated during adipogenesis, while the present invention is not limited to any particular mechanism, and an understanding of the mechanism is not necessary to practice the present invention, it is contemplated that EETs contribute to preadipocyte maturation by activating PPARγ. In this scenario, sEH could form a negative feedback mechanism to prevent further adipogenic signaling once the mature adipocyte phenotype has been established. In support of this, it has been shown that overexpression of sEH reduces PPARγ activity (Liu et al. (2005) PNAS 102:16747-16752; herein incorporated by reference in its entirety).

The precise identity of the EETs involved in adipocyte differentiation is uncertain. While the present invention is not limited to any particular mechanism, and an understanding of the mechanism is not necessary to practice the present invention, it is contemplated that 5,6-EET plays an important role considering the higher levels in comparison with the other EETs. Higher levels of 5,6-EET are in agreement with the fact that sEH effectively hydrolyzes 8,9-, 11,12-, and 14,15-EET, whereas 5,6-EET is a poor substrate for sEH (Spector et al. (2007) Am. J. Physiol. Cell Physiol. 292:C996-1012; herein incorporated by reference in its entirety). This hypothesis is supported further by data presented herein showing reduced 5,6-DHET levels in comparison with the other DHET regioisomers in the medium of undifferentiated 3T3-L1 cells. Finally, DHET levels decrease with differentiation. While the present invention is not limited to any particular mechanism, and an understanding of the mechanism is not necessary to practice the present invention, it is contemplated that this can be explained by the lower levels of EETs available for hydration. Lower levels of EETs in mature adipocytes suggest that CYP450 activity decreases upon differentiation of preadipocytes. sEH inhibitors are in preclinical trials to treat insulin resistance. Data presented herein show that sEH inhibitors increase the levels of EETs in adipose tissue simulating treatment with PPARγ agonists. Inhibiting sEH can preserve the important low levels of EETs in mature adipocytes.

Considering the role of sEH in inflammation, hypertension, and type 2 diabetes development, data presented herein show that thiazolidinediones induce sEH in vitro and in vivo, which is a surprising finding. However, while the present invention is not limited to any particular mechanism, and an understanding of the mechanism is not necessary to practice the present invention, it is contemplated that higher sEH levels may provide a negative feedback loop, preventing further adipocyte differentiation. A tight in vivo regulation of sEH-mediated EET hydrolysis in adipose tissue can be anticipated based on the fact that EETs, acting as PPARγ ligands, indirectly induce their own hydrolysis via stimulation of sEH expression. Furthermore, EET-induced aP2 (Liu et al (2005) PNAS 102:16747-167521; herein incorporated by reference in its entirety), which binds EETs, influences the metabolism, bioavailability, and activity of EETs (Spector et al. (2007) Am. J. Physiol. Cell Physiol. 292:C996-1012; herein incorporated by reference in its entirety).

sEH expression in adipose tissue is significantly lower than that observed in liver and kidney. However, although the normalized concentration of sEH in fat did not increase when animals were fed a high-fat diet, total adipose sEH activity was higher in obese vs. lean mice. Total adipose sEH activity was selectively increased during the development of obesity. Furthermore, the TZD rosiglitazone specifically induced sEH expression and activity in adipose tissue, resulting in levels similar to those in kidney. Thus, adipose tissue exhibits the greatest relative increase in sEH levels either due to consumption of a “western diet” or due to the use of PPARγ agonists. The diet-induced increase in sEH activity seen for liver reflects activation of abundant liver PPARγ, which induces sEH expression (Pinot et al. (1995) Biochem. Pharmacol. 50:501-508; herein incorporated by reference in its entirety). Activation of the different members of the PPAR family can result in increased sEH expression. Interestingly, sEH levels decrease in kidney when animals are fed a high-fat diet. In lean animals different levels of sEH mRNA were observed in perirenal and pericardial fat in comparison with epididymal and subcutaneous fat, suggesting fat depot specificity for sEH expression.

While the present invention is not limited to any particular mechanism, and an understanding of the mechanism is not necessary to practice the present invention, it is contemplated that PPARγ activates other transcription factors and/or induces lipogenic enzymes resulting in an increase in lipid signaling molecules that can induce sEH expression. Although increased levels of some DHETs formed after hydrolysis of EETs by sEH may retain biological activity (Spector et al. (2007) Am. J. Physiol. Cell Physiol. 292:C996-1012; herein incorporated by reference in its entirety), limited in vivo activity can be anticipated because DHETs are quite water soluble and rapidly conjugated. In addition, other epoxides are substrates for sEH (Greene et al. (2000) Arch. Biochem. Biophys. 376:420-432; herein incorporated by reference in its entirety). Experiments conducted during the course of developing some embodiments of the present invention show that selective sEH inhibitors, which are currently under investigation to treat hypertension and insulin resistance, are of value in treating metabolic and cardiovascular consequences of obesity.

Experiments conducted during the course of developing some embodiments of the present invention demonstrate, surprisingly, expression of sEH in adipose tissue, and more specifically in the adipocyte itself. EETs can be considered as novel adipokines, whose bioavailability can be regulated by adipose sEH. While the present invention is not limited to any particular mechanism, and an understanding of the mechanism is not necessary to practice the present invention, it is contemplated that sEH expression is upregulated by PPARγ agonists suggesting a complex reciprocal autoregulatory mechanism. As such, sEH in adipose tissue is topologically and functionally poised to influence local inflammation, lipid metabolism, and adipogenesis.

### A. PPARγ Agonists

Peroxisome proliferator-activated receptor gamma (PPARγ; also known as glitazone receptor or NR1C3) is a nuclear receptor that is encoded by the PPARC gene in humans. At least three alternatively spliced PPARC transcript variants encoding different isoforms are known.

PPARγ regulates fatty acid storage, adipocyte differentiation, and glucose metabolism. Additionally, PPAR-gamma has been implicated in the pathology of numerous diseases including obesity, diabetes, atherosclerosis and cancer. Insulin sensitizing drugs, including thiazolidinediones (TZDs or glitazones) used in the treatment of diabetes target PPARγ to lower serum glucose without increasing pancreatic insulin secretion. Genes activated by PPARγ stimulate lipid uptake and adipogenesis by fat cells. PPARγ knockout mice fail to generate adipose tissue when fed a high-fat diet (Jones et al. (2005) J. Biol. Chem. 275:33201-33204; herein incorporated by reference in its entirety). In general, members of the PPAR subfamily of nuclear receptors form heterodimers with retinoid X receptors (RXRs), and these heterodimers regulate transcription of various genes.

PPARγ ligands include the thiazolidinediones (Murphy et al. (2000) Trends Pharmacol. Sci. 21:469-474; herein incorporated by reference in its entirety), polyunsaturated fatty acids (Hertzel et al. (1998) Mol. Cell. Biochem. 188:33-39; herein incorporated by reference in its entirety), and arachidonic acid metabolites such as 15-deoxy-Δ^{12,14} prostaglandin J (Forman et al. (1995) Cell 83:803-812; Kliewer et al. (1995) Cell 83:813-819; each herein incorporated by reference in its entirety), among others. Other metabolites derived from arachidonic acid by either the lipoxygenase or cyclooxygenase have been shown to positively or negatively influence adipogenesis (Xie et al. (2006) Diabetes Obes. Metab. 8:83-93; Yan et al. (2003) J. Lipid Res. 44:424-429; each herein incorporated by reference in its entirety). The synthetic fluorosurfactant perfluorononanoic acid (PFNA), an environmental contaminant, has been found to activate both PPARα and PPARγ.

The thiazolidinediones (TZDs, also known as glitazones) were introduced in the late 1990s as an adjunctive therapy for diabetes mellitus (type 2) and related diseases (Parker (2002) Adv. Drug Deliv. Rev. 54:1173-1197; herein incorporated by reference in its entirety). PPARγ activation by TZDs results in decreased insulin resistance, modification of adipocyte differentiation, inhibition of VEGF-induced angiogenesis, decrease in leptin levels, decreased levels of certain interleukins (e.g., IL-6), and increased levels of adiponectin. With regard to insulin sensitization by these agents, activation of PPAR-gamma receptors regulates insulin-responsive gene transcription involved in glucose production, transport, and utilization, thereby reducing blood glucose concentrations and reducing hyperinsulinemia.

Three TZDs are currently or have previously been in clinical use: rosiglitazone (Avandia) (see, e.g., U.S. Pat. Nos. 5,972,973; 6,011,049; and 6,046,202; each herein incorporated by reference in its entirety), pioglitazone (Actos) (see, e.g., U.S. Pat. No. 4,687,777; herein incorporated by reference in its entirety), and troglitazone (Rezulin) (see, e.g., U.S. Pat. Nos. 5,457,109; 5,478,852; and 5,708,012; each herein incorporated by reference in its entirety). All are derivatives of the parent compound thiazolidinedione. Troglitazone was removed from the U.S. market in 2000 due to increased incidence of drug-induced liver toxicity (hepatitis). Experimental or preclinical TZDs include but are not limited to MCC-555, rivoglitazone, ciglitazone, and englitazone.

The withdrawal of troglitazone has led to concerns of the other thiazolidinediones also increasing the incidence of hepatitis and potential liver failure, an approximately 1 in 20,000 individual occurrence with troglitazone. Because of this, the FDA recommends two to three month tests of liver enzymes for the first year of thiazolidinedione therapy to check for this rare but potentially catastrophic complication. Thus far, newer thiazolidinediones (rosiglitazone and pioglitazone) have not shown higher incidence.

The main side effect of all thiazolidinediones is water retention, leading to edema. In some patients, significant water retention leads to a decompensation of potentially previously unrecognized heart failure. Therefore, thiazolidinediones are prescribed with both caution and patient warnings about the potential for water retention/weight gain, especially in patients with decreased ventricular function (NYHA grade III or IV heart failure).

The US Food and Drug Administration issued an alert on May 21, 2007 to patients and health care professionals of rosiglitazone potentially causing an increased risk of myocardial infarction (MI) and heart-related deaths following the online publication of a meta-analysis. In a large-scale trial, the RECORD (Rosiglitazone Evaluated for Cardiac Outcomes and Regulation of Glycaemia in Diabetes) study, cardiovascular outcomes were assessed after adding rosiglitazone to metformin or sulfonylurea regimens for type 2 diabetes mellitus (Home et al. (2009) Lancet 199:207-214). The study was a multicenter, open-label trial that included 4447 patients with mean HbA1c of 7.9%. Follow-up of the 2 drug combinations took place over 5-7 years. No difference was observed between the 2 groups for cardiovascular death, myocardial infarction, or stroke.

In the study, 61 patients who received rosiglitazone experienced heart failure that caused either hospital admission or death, compared with 29 patients in the active control group (hazard ratio [HR] 2.10, 1.35-3.27, risk difference per 1000 person-years 2.6, 1.1-4.1). Noncardiovascular adverse effects included increased upper and distal lower limb fracture rates, particularly in women. At 5 years, mean HbA1c was lower in the rosiglitazone group compared with the active control group. The investigators concluded that the use of rosiglitazone for type 2 diabetes mellitus increases the risk of heart failure. The study's results also indicated that the risk for select fractures, particularly in women, is increased as well.

Rosiglitazone is indicated as monotherapy and in conjunction with sulfonylureas and/or metformin and insulin. It is an insulin sensitizer with major effect on stimulation of glucose uptake in skeletal muscle and adipose tissue, and it lowers plasma insulin levels. Rosiglitazone may preserve beta cell function, and has positive effects on vasculature and inflammation. It also induces changes in LDL and HDL particle size. Currently recommended dosing for rosiglitazone in adult humans is 4-8 mg/d orally once a day or divided twice a day.

Pioglitazone improves target cell response to insulin without increasing insulin secretion from pancreas. It increases insulin-dependent glucose use in skeletal muscle and adipose tissue, and lowers triglycerides more than rosiglitazone, probably related to an effect on PPAR alpha. Currently recommended dosing for pioglitazone in adult humans is 15-30 mg by mouth once a day, which may be increased, but not to exceed 45 mg/d.

### B. Soluble Epoxide Hydrolase Inhibitors

Epoxide hydrolases (sEH) add water to epoxides (three-membered cyclic ethers), and the mammalian sEH is highly selective for epoxides of fatty acids, with the regioisomeric epoxides of arachidonic acid or epoxyeicosanoids (EETs) being particularly good substrates. EETs themselves cause vasodilation, reduce blood pressure, and have anti-inflammatory (Schmelzer et al. (2006) PNAS 103:13646-13651; Inceoglu et al. (2007) Prostaglandins Other Lpid Mediat. 82:42-49; Schmelzer et al. (2005) PNAS 102:9772-9777; each herein incorporated by reference in its entirety) and analgesic (Inceoglu et al. (2006) Life Sci. 79L2311-2319; Schmelzer et al. (2006) PNAS 103:13646-13651; Inceoglu et al. (2007) Prostaglandins Other Lipid Mediat. 82:42-49; each herein incorporated by reference in its entirety) properties. The action of sEH on EET substrates results in conversion of EETs to inactive forms (dihydroxyeicosatrienoic acids or DHETs); therefore, the inhibition of sEH activity results in an increase in circulating EETs, which can in turn cause reduction of blood pressure (Chiamvimonvat et al. (2007) J. Cardiovasc. Pharmacol. 50:225-237; herein incorporated by reference in its entirety). Therefore, sEH inhibitors (sEHIs) are of particular interest for treatment of numerous diseases including hypertension (see, e.g., U.S. Pat. No. 6,351,506; herein incorporated by reference in its entirety). No sEHIs have been approved for clinical use at present, although Arete Therapeutics Inc. has completed Phase I trials with sEH inhibitor AR9281, and Phase II trials are currently underway.

Mammalian sEH exists as a homodimer of two 60-kDa monomers, each with an N-terminal domain possessing lipid phosphatase activity and joined by a proline-rich bridge to a C-terminal domain with a classical α/β-hydrolase fold and possessing epoxide hydrolase activity (Chiamvimonvat et al. (2007) J. Cardiovasc. Pharmacol. 50:225-237; herein incorporated by reference in its entirety). Structural elucidation of human and mouse sEH enzymes (Argiriadi et al. (2000) J. Biol. Chem. 275:15265-15270; Gomez et al. (2004) Biochem. 43:4716-4723; Gomez et al. (2006) Protein Sci. 15:58-64; each herein incorporated by reference in its entirety) allowed the development of assays that facilitated design and screening efforts for sEHIs (for a review, see, e.g., Chiamvimonvat et al. (2007) J. Cardiovasc. Pharmacol. 50:225-237; herein incorporated by reference in its entirety).

The first-identified classes of sEHIs included chalcones (Mullin et al. (1982) Arch. Biochem. Biophys. 216:423-439; herein incorporated by reference in its entirety) and glycidols (Dietze et al. (1991) Biochem. Pharmacol. 42:1163-1175; herein incorporated by reference in its entirety). However, the poor pharmacokinetic profiles of these classes limit their utility in vivo. Determination of the catalytic mechanism of sEH allowed design of transition state mimic inhibitors (Morisseau et al. (2005) Ann. Rev. Pharmacol. Toxicol. 45:311-333; herein incorporated by reference in its entirety), leading to identification of urea, amide, and carbamate inhibitors of sEH (Morisseau et al. (1999) PNAS 96:8849-8854; herein incorporated by reference in its entirety). These classes of inhibitors were further refined by structure-activity screening studies (Nakagawa et al. (2000) Bioorg. Med. Chem. 8:2663-2673; McElroy et al. (2003) J. Med. Chem. 46:1066-1080; each herein incorporated by reference in its entirety). Classical structure-activity relationship studies led to identification of general structures of sEHIs such as a central pharmacophore A-C(═O)═B, a left R group such as adamantine or trifluoromethoxyphenyl, a first linker, a polar secondary pharmacophore Y, sometimes a second linker, and a terminal or tertiary pharmacophore Z (see, e.g., Chiamvimonvat et al. (2007) J. Cardiovasc. Pharmacol. 50:225-237; herein incorporated by reference in its entirety). Compounds in this series include but are not limited to DCU, AUDA, APAU, and AEPU. In some embodiments, the general structures of sEHIs may be summarized as a urea, carbamate, or amide centrapharmacophore at A-C(═O)—B with relatively hydrophobic R, linker 1, and linker 2 groups wherein either A or B needs to have a hydrogen bond donor in either a linear or cyclic structure; a polar residue such as an ester, sulfonamide, ether, or carbamate exists at Y; and a polar group exists at Z (Chiamvimonvat et al. (2007) J. Cardiovasc. Pharmacol. 50:225-237; herein incorporated by reference in its entirety). Further designs resulted in the development of additional sEHIs such as TCUB, TPAU, APAU, ACUB, t-ACUB, and TUPS, some of which are suitable for oral administration (Chiamvimonvat et al. (2007) J. Cardiovasc. Pharmacol. 50:225-237; herein incorporated by reference in its entirety). More recently, non-urea inhibitors of sEH have been identified (Xie et al. (2009) Bioorgan. Med. Chem. Lett. 19:2354-2359; Anandan et al. (2009) Bioorg. Med. Chem. Lett. 19:1066-1070; each herein incorporated by reference in its entirety).

Soluble epoxide hydrolase inhibitors are further described in, e.g., U.S. patent application Ser. Nos. 10/328,495; 10/056,284; 10/694,641; 10/817,334; 10/970,373; 10/815,425; 11/189,964; 11/240,444; 11/234,845; 11/330,033; 11/256,685; 11/566,171; 11/685,674; 11/961,881; 12/063,653; 11/995,379; 11/719,092, each herein incorporated by reference its entirety.

The dose, frequency and timing of such administration of any sEHI depends in large part on the selected therapeutic agent, the nature of the condition being treated, the condition of the subject including age, weight and presence of other conditions or disorders, the formulation being administered and the discretion of the attending physician. Preferably, the compositions and compounds of the invention and the pharmaceutically acceptable salts thereof are administered via oral, parenteral, subcutaneous, intramuscular, intravenous or topical routes. Generally, the compounds are administered in dosages ranging from about 2 mg up to about 2,000 mg per day, although variations will necessarily occur depending on the disease target, the patient, and the route of administration. Dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day. The dosage employed for the topical administration will, of course, depend on the size of the area being treated.

### C. Disease States and Conditions

The present invention finds use in the treatment of subjects with diabetes or a diabetes-related condition involving e.g., impaired glucose tolerance, impaired insulin sensitivity, impaired insulin production. Such conditions and disease states include diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus.

The present invention finds use in the treatment of subjects who are overweight or obese, who may also have or may be at risk of having diabetes or a diabetes-related condition as listed supra, or other conditions such as hypertension or cardiovascular disease. The most widely accepted clinical definition of obesity is the World Health Organization (WHO) criteria based on BMI. Under this convention for adults, grade 1 overweight (commonly and simply called overweight) is a BMI of 25-29.9 kg/m². Grade 2 overweight (commonly called obesity) is a BMI of 30-39.9 kg/m². Grade 3 overweight (commonly called severe or morbid obesity) is a BMI greater than or equal to 40 kg/m². The surgical literature often uses a different classification to recognize particularly severe obesity. In this setting, a BMI greater than 40 kg/m² is described as severe obesity, a BMI of 40-50 kg/m² is termed morbid obesity, and a BMI greater than 50 kg/m² is termed super obese. The definition of obesity in children involves BMIs greater than the 85th (commonly used to define overweight) or the 95th (commonly used to define obesity) percentile, respectively, for age-matched and sex-matched control subjects. Secondary causes of obesity include but are not limited to hypothyroidism, Cushing syndrome, insulinoma, hypothalamic obesity, polycystic ovarian syndrome, genetic syndromes (eg, Prader-Willi syndrome, Alström syndrome, Bardet-Biedl syndrome, Cohen syndrome, Börjeson-Forssman-Lehmann syndrome, Fröhlich syndrome), growth hormone deficiency, oral contraceptive use, medication-induced obesity (e.g., phenothiazines, sodium valproate, carbamazepine, tricyclic antidepressants, lithium, glucocorticoids, megestrol acetate, thiazolidine diones, sulphonylureas, insulin, adrenergic antagonists, serotonin antagonists (especially cyproheptadine), eating disorders (especially binge-eating disorder, bulimia nervosa, night-eating disorder), hypogonadism, pseudohypoparathyroidism, and obesity related to tube feeding.

The present invention finds use in the treatment of congestive heart failure (CHF). CHF is an imbalance in pump function in which the heart fails to adequately maintain the circulation of blood. The most severe manifestation of CHF, pulmonary edema, develops when this imbalance causes an increase in lung fluid secondary to leakage from pulmonary capillaries into the interstitium and alveoli of the lung. CHF can be categorized as forward or backward ventricular failure. Backward failure is secondary to elevated systemic venous pressure, whereas left ventricular failure is secondary to reduced forward flow into the aorta and systemic circulation. Furthermore, heart failure can be subdivided into systolic and diastolic dysfunction. Systolic dysfunction is characterized by a dilated left ventricle with impaired contractility, whereas diastolic dysfunction occurs in a normal or intact left ventricle with impaired ability to relax and receive as well as eject blood. The New York Heart Association's functional classification of CHF is one of the most useful. Class I describes a patient whose normal physical activity is not limited by symptoms. Class II occurs when ordinary physical activity results in fatigue, dyspnea, or other symptoms. Class III is characterized by a marked limitation in normal physical activity. Class IV is defined by symptoms at rest or with any physical activity. CHF and/or pulmonary edema may be caused by coronary artery disease (e.g., secondary to loss of left ventricular muscle), ongoing ischemia, decreased diastolic ventricular compliance; hypertension, valvular heart disease, congenital heart disease, other cardiomyopathies, myocarditis, infectious endocarditis, pregnancy, and hyperthyroidism. CHF is often precipitated by cardiac ischemia or dysrhythmias, cardiac or extracardiac infection, pulmonary embolus, physical or environmental stresses, changes or noncompliance with medical therapy, dietary indiscretion, or iatrogenic volume overload.

The present invention finds use in the treatment of edema, which is an abnormal accumulation of fluid beneath the skin, or in one or more cavities of the body. Causes of edema which are generalized to the whole body can cause edema in multiple organs and peripherally. For example, severe heart failure (CHF, as described supra) can cause pulmonary edema, pleural effusions, ascites and peripheral edema, the last of which effects can also derive from less serious causes. Organ-specific edema types include cerebral edema, pulmonary edema, pleural efflusions, corneal edema, periorbital edema, cutaneous edema, myxedema, edema of the feet, and lymphedema.

### D. Methods of Combined Therapy

Resolution or prevention of PPARγ-agonist-induced conditions (e.g., edema, congestive heart failure) is a major clinical challenge in TZD therapy. Compositions and methods of the present invention provide means of ameliorating this problem by effectively administering a combined therapy approach. However, it should be noted that traditional combination therapy may be employed in combination with the compositions of the present invention. For example, in some embodiments of the present invention, TZD compositions may be used before, after, or in combination with the traditional therapies.

To treat a subject using the methods and compositions of the present invention in combination therapy, one contacts a “target” cell with the compositions described herein and at least one other agent. These compositions are provided in a combined amount effective to have a therapeutic effect the cell. This process may involve contacting the cells with multiple agent(s) or factor(s) at the same time. This may be achieved by contacting the cell with a single composition or pharmacological formulation that includes both agents, or by contacting the cell with two distinct compositions or formulations, at the same time, wherein one composition includes, for example, an expression construct and the other includes a therapeutic agent.

Alternatively, PPARγ agonist treatment may precede or follow the other agent treatment (e.g., inhibition of soluble epoxide hydrolase) by intervals ranging from minutes to weeks. In embodiments where the PPARγ agonist and sEH inhibitor are applied separately to the cell, one would generally ensure that a significant period of time did not expire between the time of each delivery, such that the PPARγ agonist and sEH inhibitor composition would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent are utilized. Various combinations may be employed, where the PPARγ agonist composition is “A” and the sEH inhibitor is “B”, as exemplified below:

### A/B/A, B/A/B, B/B/A, A/A/B, B/A/A, A/B/B, B/B/B/A, B/B/A/B,

### A/A/B/B, A/B/A/B, A/B/B/A, B/B/A/A, B/A/B/A, B/A/A/B, B/B/B/A,

### A/A/A/B, B/A/A/A, A/B/A/A, A/A/B/A, A/B/B/B, B/A/B/B, B/B/A/B.

Other combinations are contemplated. Again, to achieve desired therapeutic effect, both agents are delivered to a cell in a combined amount effective to promote desired therapeutic outcome (e.g., increased insulin sensitivity, decreased fluid retention, increased natriuresis).

In some embodiments of the invention, one or more compounds of the invention and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times which overlap. Each additional active agent can be administered separately, in any appropriate form and by any suitable route. In other embodiments, the compound is administered before, concurrently or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, a PPARγ agonist and an sEH inhibitor (and optionally additional active agents) are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reduce the development of resistance to one or more of the therapies, avoid or reduce the side effects of one or more of the therapies, and/or improve the efficacy of the treatment.

In other examples, one or more compounds of some embodiments of the present invention and optionally an additional active agent are administered in a cycle of less than about 3 weeks, about once every two weeks, about once every 10 days or about once every week. One cycle can comprise the administration of a first active agent and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

### EXAMPLES

The following examples are provided in order to demonstrate and further illustrate certain preferred embodiments and aspects of the present invention and are not to be construed as limiting the scope thereof.

### Example 1

### Expression and Regulation of Soluble Epoxide Hydrolase in Adipose Tissue

### Methods and Procedures

### Animals for Proteomic Analysis

Adult (6 week-old) male C57BL/6J mice were purchased from the Jackson Laboratory (Bar Harbor, Me.). Mice were housed in a pathogen-free barrier facility (12 h light/12 h dark cycle). For the proteomic analysis 5 animals received a regular, standard fat diet (SFD) for 20 weeks (Diet 5001; LabDiet, Richmond, Ind.) in which 12% of the calories were derived from fat and 5 animals received a high fat diet (HFD) for 20 weeks (Diet TD 88137; Harlan Teklad, Madison, Wis.) in which 42% of total calories were derived from fat. Mice were weighed every two weeks. At the beginning and the end of the feeding period, body composition was determined using a Minispec Model mq 7.5 (7.5 mHz) (Brucker Optics, Billerica, Mass.) and the animals were sacrificed under anaesthesia with isoflurane (Baxter, Deerfield, Ill.). The epididymal fat pads, livers and kidneys were harvested and weighed; one part of the tissues was snap-frozen in liquid nitrogen and stored at −80° C. for further analysis while another part was fixed in 10% formalin for immunohistochemistry. To evaluate expression of sEH in different adipose depots, 6 week-old C57BL/6J mice were fed the HFD or SFD for 13 weeks; epididymal fat pads, subcutaneous fat pads, perirenal fat pads and pericardial fat pads were collected and stored at −80° C. These studies were initiated at Vanderbilt University and completed at Northwestern University. All animal protocols were approved by the Vanderbilt University Institutional Animal Care and Use Committee and the Northwestern University Institutional Animal Care and Use Committee.

### Protein Extraction

Protein was isolated from epididymal adipose tissue, liver and kidney (200-400 mg). For proteomic analysis and western blotting, the samples were homogenized on ice in 1 mL lysis buffer (20 mmol/L HEPES, 50 mmol/L NaCl, 10% glycerol, 1% Triton-X 100, pH 7.4) in the presence of protease inhibitor cocktail (Complete Mini, Roche Diagnostics, Indianapolis, Ind.). After incubation for 1 hr at 4° C., samples were centrifuged at 4° C. for 20 min at 13,000 rpm. The aqueous layer containing the protein was collected. For the sEH activity assay, epididymal fat, liver and kidney samples were homogenized in 3 mL of chilled 0.1 mol/L sodium phosphate buffer pH 7.4, containing 1 mmol/L EDTA, PMSF and DTT; the homogenate was centrifuged at 11,000 rpm for 20 min at 4° C.; the supernatant solution was used as the enzyme extract. The extracts were snap-frozen in liquid nitrogen and stored at −80° C. until used. For each extraction method, protein concentration was determined using a BCA assay following the manufacturer's instructions (Pierce, Rockford, Ill.).

### Analysis of the Adipose Proteome

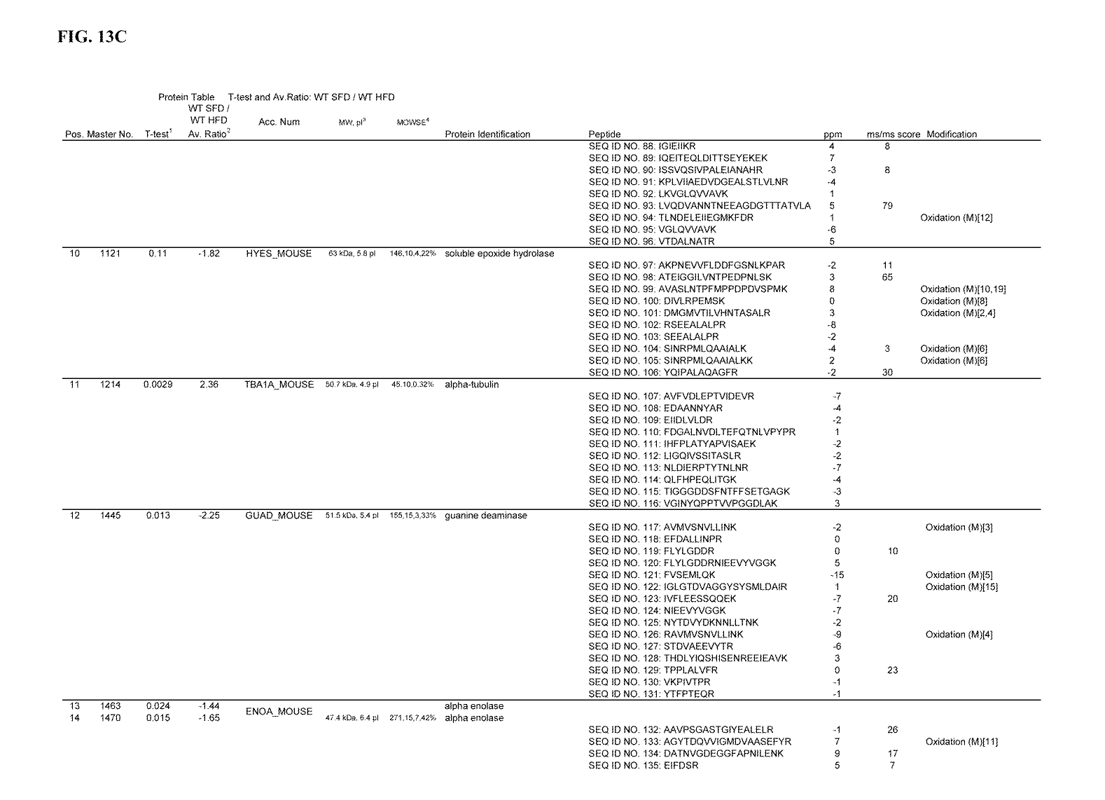

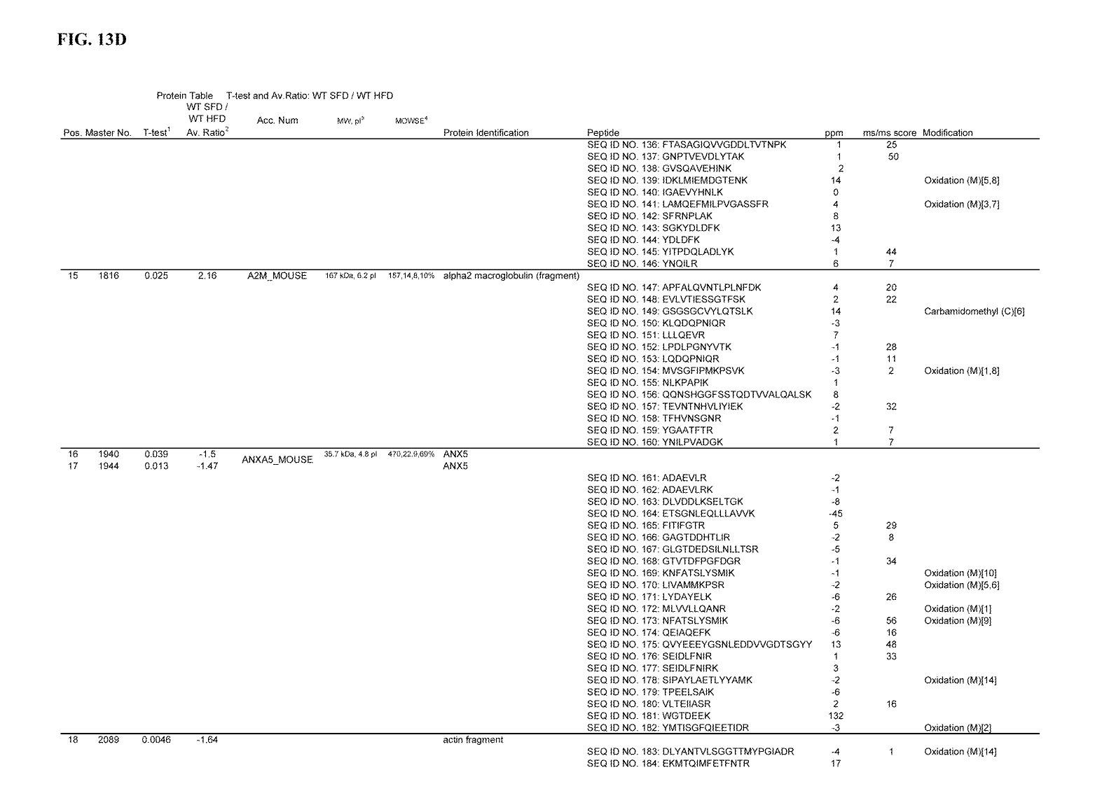

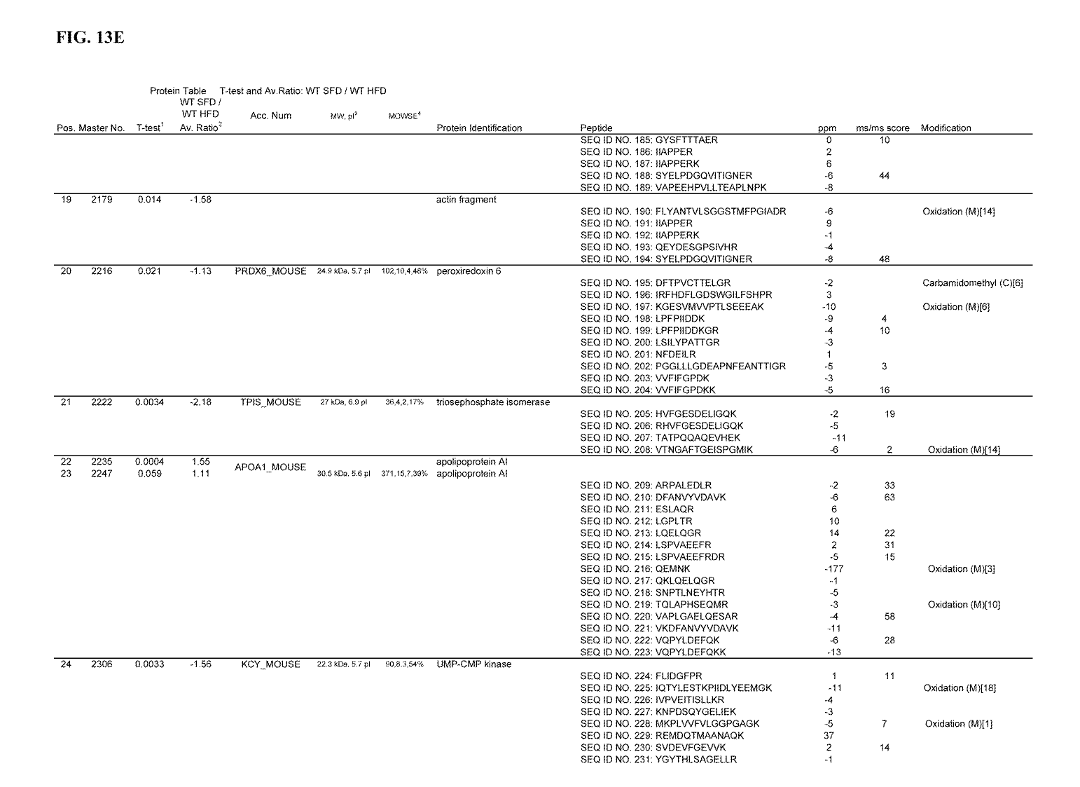

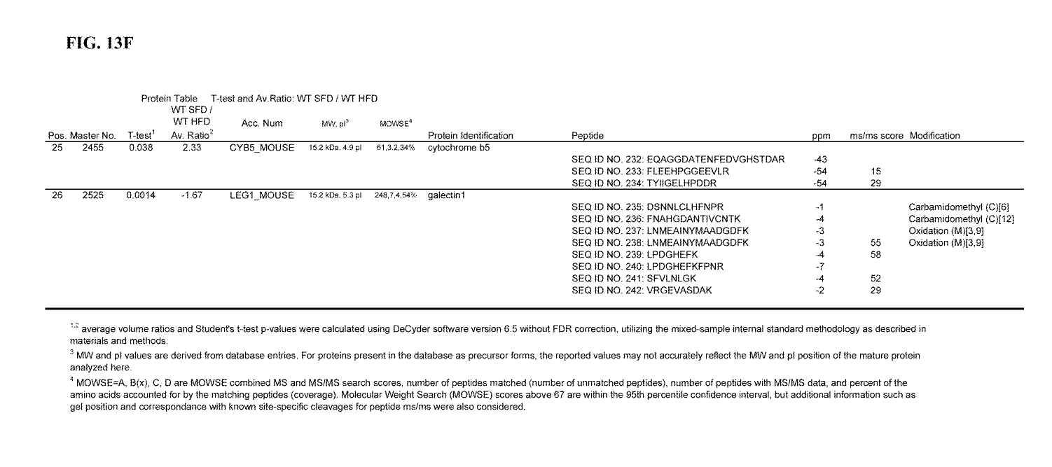

Differences in protein levels were analyzed using proteomics technology, as described (Friedman et al. (2004) Proteomics 4:793-811; herein incorporated by reference in its entirety). Protein samples (n=3) were Cy-dye labeled using either Cy3 or Cy5 so that samples from each condition (SFD vs HFD) received each labeling procedure to exclude a potential effect of labeling efficiency. A mix of all 6 samples was labeled using Cy2. Subsequently, 2-D gel electrophoresis was performed using a pH 4-7 immobilized pH gradient. Three gels were run each containing a Cy3 and Cy5 labeled sample so that each gel contained a sample from both the SFD and HFD group. Each of the 3 gels also contained the Cy2 labeled mixture as internal standard. The Cy2, Cy3 and Cy5 components of each gel were individually imaged using mutually exclusive excitation/emission wavelengths. Finally, the gels were incubated in Sypro Ruby to ensure accurate protein excision, as the molecular weight and hydrophobicity of the Cy-dyes can influence protein migration during SDS-PAGE. DeCyder software (GE Healthcare, Piscataway, N.J.) was used for simultaneous comparison of abundance changes across all 3 gels. The DeCyder biological variation analysis module was then used to simultaneously match all 9 protein-spot maps from the 3 gels, and using the Cy3:Cy2 and Cy5:Cy2 differential in-gel analysis ratios, calculate average abundance changes and Student's t-test p-values for the variance of these ratios for each protein-pair. Fold abundance changes are reported, whereby a fold increase is calculated directly from the volume ratio, and the fold decrease =1/volume ratio (see FIGS. 13A-F).

Proteins of interest were robotically excised, equilibrated with 100 mmol/L ammonium bicarbonate for 20 min and dehydrated with two 10-min incubations with 100% acetonitrile in a 96-well plate format using Ettan Spot Picker and Digester workstations (GE Healthcare). Dehydrated gel plugs were manually digested in-gel with 10 μL, porcine modified trypsin protease (Promega, Madison, Wis.) in 25 mmol/L ammonium bicarbonate for 2 hrs at 37° C. Tryptic peptides were then extracted from the gel plugs in two cycles of 60% acetonitrile, 0.1% trifluoroacetic acid using the Ettan Digester workstation and dried by vacuum centrifugation. Peptides were reconstituted in 10 μL, 0.1% trifluoroacetic acid, and manually desalted/concentrated into 2 μL, 60% acetonitrile, 0.1% trifluoroactetic acid using C18 ZipTip pipette tips (Millipore Corp., Billerica, Mass.). A 0.5 μL, volume of the eluate was analyzed using MALDI-TOF and tandem TOF/TOF mass spectrometry using a Voyager 4700 (Applied Biosystems, Foster City, Calif.). Ions specific for each sample were used to interrogate murine sequences entered in the SWISS-PROT and NCBInr databases using the MASCOT and ProFound database search algorithms, respectively. Protein identifications from MALDI-TOF peptide mass maps are based on the masses of the tryptic peptides. Tandem mass spectrometry (MALDI-TOF/TOF) was used to generate limited amino acid sequence information on selected ions if additional confirmation was required.

### Real-Time Quantitative PCR and Reverse Transcriptase PCR

RNA isolation, reverse transcription, and real-time quantitative PCR were performed as described previously (De Taeye et al. (2007) Am. J. Physiol. Endocrinol. Metabol. 293:E713-725; herein incorporated by reference in its entirety), with the primer sequences given in Table 1.

TABLE 1
Real-time quantitative PCR primers.

| Target | Primer sequence 5′ to 3′ |
|---|---|
| SEQ ID NO. 243: sEH | CCATAAGTCAAATATTCAGCCAAG |
| sense | |
| SEQ ID NO. 244: sEH | TATCAGGAAGTCAAAGTGTTGG |
| antisense | |
| SEQ ID NO. 245: aP2 | GCGTGGAATTCGATGAAATCA |
| sense | |
| SEQ ID NO. 246: aP2 | CCCGCCATCTAGGGTTATGA |
| antisense | |
| SEQ ID NO. 247: Pref-1 | AATAGACGTTCGGGCTTGCA |
| sense | |
| SEQ ID NO. 248: Pref-1 | GGAGCATTCGTACTGGCCTTT |
| antisense | |
| SEQ ID NO. 249: Leptin | CCCCTCAGATCCTCCAAAAT |
| sense | |
| SEQ ID NO. 250: Leptin | AACCCTGCTTGCAGTCTATT |
| antisense | |
| SEQ ID NO. 251 F4/80 | TTTCCTCGCCTGCTTCTTC |
| sense | |
| SEQ ID NO. 252: F4/80 | CCCGTCTCTGTATTCAACC |
| antisense | |
| SEQ ID NO. 253: β-actin | GATTACTGCTCTGGCTCCTAGCA |
| sense | |
| SEQ ID NO. 254: β-actin | GCCACCGATCCACACAGAGT |
| antisense | |

Reverse transcriptase PCR was performed on cDNA created as described (De Taeye et al. (2007) Am. J. Physiol. Endocrinol. Metabol. 293:E713-725; herein incorporated by reference in its entirety). PCR products were analyzed on a 1% agarose gel followed by ethidium bromide staining. Reaction conditions and primers used are described (De Taeye et al. (2007) Am. J. Physiol. Endocrinol. Metabol. 293:E713-725; herein incorporated by reference in its entirety).

### Western Blotting

Proteins were separated using NuPAGE 4-12% Bis-Tris gels (Invitrogen Corp., Carlsbad, Calif.) and transferred to Immobilon-P membranes (Millipore Corp.). After blocking, protein immunoblot analysis was performed using the appropriate primary antibodies (rabbit antiserum against mouse sEH, 1:20,000, rabbit polyclonal antibody for β-actin, 1:5000 (Abcam Inc., Cambridge, Mass.), rabbit polyclonal antibody for perilipin, 1:1000 (Abcam Inc.)) followed by horseradish peroxidase-conjugated anti-rabbit IgG antibody (1:10,000) (Santa Cruz Biotechnology Inc., Santa Cruz, Calif.). Detection was done using the ECL Plus kit following manufacturer's instructions (GE Healthcare or Bio-Rad, Hercules, Calif.). Recombinant mouse sEH was used as a positive control in preliminary experiments to validate the specificity of the rabbit antiserum against mouse sEH.

### Analysis of sEH Activity

sEH activity was determined in liver, kidney and epididymal fat pad, as described (Morisseau et al. (2007) Curr. Protoc. Toxicol. 44:4.23.1-4.23.18; herein incorporated by reference in its entirety), using [³H]trans-stilbene oxide (t-SO). A 5 mmol/L t-SO solution (10,000 cpm/μL) in EtOH was prepared with cold t-SO (Sigma-Aldrich, St. Louis, Mo.) and [³H]t-SO (American Radiolabeled Chemicals, St. Louis, Mo.). One μL of this 5 mmol/L solution was added to 100 μL tissue extract, diluted 50-, 20-, or 5-fold in 0.1 mol/L sodium phosphate buffer, pH 7.4, containing 0.1 mg/mL bovine serum albumin for liver, kidney and fat, respectively. After incubation at 30° C. for 10, 15 and 60 min for liver, kidney and fat, respectively, the reaction was stopped by addition of 250 μL of isooctane or hexanol. These reagents extracted the remaining epoxide or epoxide and diol, respectively, from the aqueous phase, and the quantity of radioactive diol present in the aqueous phase was measured by liquid scintillation counting. Each assay was performed in triplicate.

### Immunohistochemistry

Tissues for immunohistochemistry were fixed in 10% neutral, phosphate buffered formalin for 24 to 48 hrs and paraffin-embedded. Subsequently the paraffin embedded tissues were processed in six μm sections. sEH antigen was localized using rabbit anti-mouse sEH IgG fraction (1:2000). For detection ready-to-use biotinylated secondary antibodies against rabbit IgGs were used in combination with ready-to-use Streptavidin-HRP and AEC for detection (all from BioGenex, San Ramon, Calif.).

Separation of Adipocytes and Stromal Cells from Adipose Tissue

Three lean, male 6 week-old C57BL/6J mice, fed a SFD were sacrificed as described above and their epididymal fat pads were collected. In addition, part of the edidiymal fat pads of 4 of the male 6 week-old C57BL/6J mice that were fed the above described HFD for 13 weeks was used. From each mouse, 300-500 mg of fat was collected in DMEM containing 1% BSA and minced into fine pieces. The adipose tissue was digested using type I collagenase (1 mg/mL; Worthington, Lakewood, N.J.) for 2 hrs at room temperature. The digested tissue was filtered through a 100 μm cell strainer (BD Falcon, BD Biosciences, Bedford, Mass.) followed by centrifugation at 300×g. The infranatant (liquid phase under the floating adipocytes) was collected using a syringe with needle. Fat cells were washed 3 times with PBS, followed by centrifugation and collection of infranatant. Stromal cells were isolated by centrifuging the infranatant at 2000×g. Stromal cells were washed 2 times with PBS. Total RNA was isolated from both fractions using Trizol reagent (Invitrogen Corp.) following manufacturer's instructions. Leptin, F4/80 and sEH mRNA levels were quantified using real-time quantitative PCR.

### Culture and Differentiation of 3T3-L1 Cells

3T3-L1 cells were purchased from American Type Culture Collection (Rockville, Md.) and cultured in DMEM (1 mg/mL glucose) supplemented with 10% fetal bovine serum and antibiotic-antimycotic solution (standard medium) in 100-mm culture dishes. For differentiation to adipocytes, cells were cultured in the appropriate plates (6-well or 100-mm), and when confluent (day 0), the standard medium was changed to differentiation medium (DMEM (4.5 mg/mL glucose) supplemented with 10% fetal bovine serum and antibiotic-antimycotic solution additionally supplemented with 0.5 mmol/L isobutylmethylxanthine, 1.0 μmol/L dexamethasone and 10 μg/mL insulin) (Sigma-Aldrich). The differentiation medium was changed at days 2 and 4. Differentiation was considered to be complete at day 7. At this time >95% of the cells had accumulated fat droplets as shown by oil red O (Sigma-Aldrich). Before treatment, cells were incubated for 24 hrs in serum-free standard medium containing 1% bovine serum albumin (starvation medium). Subsequently cells were treated with troglitazone (Sigma-Aldrich) in starvation medium at concentrations and for the time indicated for the different experiments. After treatment, cells were washed with PBS and collected in Trizol (RNA analysis) (Invitrogen Corp.) or lysis buffer (protein analysis).

### Oxylipin Extraction and Analysis of 3T3-L1 Cells and Cell Culture Medium

Samples stored at −80° C. were thawed at room temperature and spiked with 10 μL 500 nM surrogate solution (Luria et al. (2007) J. Biol. Chem. 282:2891-2898; herein incorporated by reference in its entirety) and antioxidant solution (BHT:EDTA, 17 mg/mL) and oxylipins were extracted by solid phase extraction. Prior to that, cell pellets were manually homogenized in guanidinium thiocyanate solution (4M; Invitrogen Corp.) to elute nucleic acids from samples. Solid phase extraction was performed with Waters Oasis HLB cartridges (Waters Corp, Milford, Mass.) and the cartridges were washed with 6 mL of a 5% methanol (MeOH)/water solution containing 0.1% acetic acid. The analytes were eluted with 0.5 ml MeOH followed by elution with 2 mL of ethyl acetate. The eluate was evaporated with 10 μL of a trap solution (30% glycerol in MeOH) by vacuum centrifugation (Speed-Vac). Prior to analysis, analytes were reconstituted by adding 50 μL of 200 nM of the internal standard solution as described previously (Luria et al. (2007) J. Biol. Chem. 282:2891-2898; herein incorporated by reference in its entirety) to quantify recovery of surrogate standards so that recovery of 70% and up was accepted for analysis. Blank samples were PBS samples spiked with analytical surrogates and antioxidants for calculation of limit of detection. All extracted samples were quantified using HPLC-MS/MS as previously described (Luria et al. (2007) J. Biol. Chem. 282:2891-2898; herein incorporated by reference in its entirety). Epoxides and diol analytes were quantified using the internal standard method and seven point calibration curve fit with 1/x weighted either linear or quadratic curves (r²>0.997).

Treatment of Animals with Rosiglitazone

Ten adult (6 week-old) male C57BL/6J mice were purchased from the Jackson Laboratory. Mice were housed in a pathogen-free barrier facility (12 h light/12 h dark cycle). All animals received the high fat diet (HFD) for 12 weeks (Diet TD 88137; Harlan Teklad, Madison, Wis.). Mice were weighed every 4 weeks. After 12 weeks blood was collected after a 6 hour fast and plasma glucose levels were analyzed using the Accu-Check glucose meter. Subsequently, mice were treated with rosiglitazone (10 mg/kg/day in 1% carboxymethylcellulose in water) or vehicle by gavaging for 10 days. On day 10 the animals were subjected to an intraperitoneal glucose tolerance test (IPGTT) after a 16 hr overnight fast. Plasma glucose levels were determined at 0, 15, 30, 60 and 120 min using an Accu-Check glucose meter; at each time point 10 μl blood was collected in EDTA-coated tubes (Microvette CB300, Sarstedt, Newton, N.C.) for determination of insulin levels using an ELISA specific for mouse insulin (Alpco Diagnostics, Salem, N.H.). After the IPGTT, mice received a final treatment with rosiglitazone or vehicle (day 11) and were euthanized the next day (day 12) after a 6 hour fast under anaesthesia with isoflurane (Baxter). The epididymal fat pads, subcutaneous fat pads, liver and kidneys were harvested and stored at −80° C. for further analysis. All animal protocols were approved by the Northwestern University Institutional Animal Care and Use Committee.

### Statistical Analysis

Results are expressed as mean±SEM. Comparisons between groups were carried out by unpaired Student's t-test or one-way ANOVA followed by Bonferroni's post test where appropriate. For repetitive measurements two-way ANOVA was used. Statistical analysis was performed using GraphPad Prism 4 (San Diego, Calif.).

### The Adipose Proteome—Identification of Soluble Epoxide Hydrolase

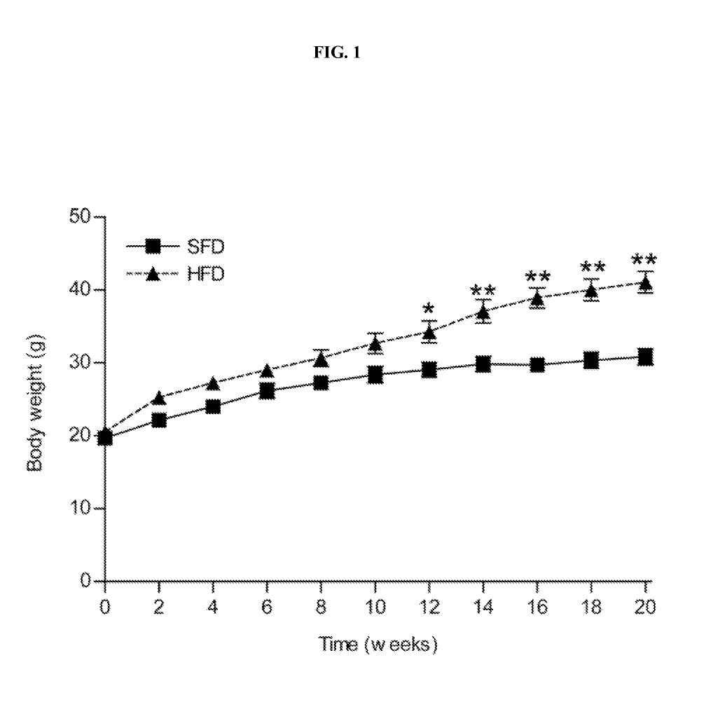

While the present invention is not limited to any particular mechanism, and an understanding of the mechanism is not necessary to practice the present invention, it is contemplated that novel or previously unrecognized proteins are expressed in visceral fat that contribute to the cardiovascular consequences of obesity. Therefore, the adipose proteome isolated from epididymal fat pads from C57BL/6J animals fed a HFD or a SFD for 20 weeks, was analyzed (n=3). Whereas initial body weights did not differ (p>0.05), the animals fed a high-fat diet were significantly heavier than their lean counterparts at the end of the feeding period (p<0.001; Table 2 infra and FIG. 1). This was reflected in the % total adipose tissue (p=0.003; Table 2) and was in agreement with a 6-fold higher epididymal fat pad weight in the HFD group (p=0.0003; Table 2).

TABLE 2
Obesity parameters for C57BL/6J animals in the study.

| | SFD | HFD |
|---|---|---|
| Initial weight (g) | 19.7 ± 0.6 | 20.5 ± 0.5 |
| End weight (g) | 30.8 ± 1.1 | 41.1 ± 1.5 |
| Total adipose tissue (%)* | 8.7 ± 0.7 | 32.2 ± 1.1 |
| Epididymal fat pad weight (g)* | 0.45 ± 0.05 | 2.65 ± 0.19 |

Data are mean ± s.e.m. SFD, standard fat diet; HFD, high fat diet.
*P < 0.01; n = 5.

From approximately 1,500 resolved proteins, 19 differentially expressed proteins were identified (selection based on p<0.05 or fold change ≧1.5 (see FIGS. 13a-f supra)). Among proteins that are typically detected in proteomic experiments such as kininogen, apolipoprotein AI and α₂-macroglobulin, sEH was selected for further investigation because of its roles in fatty acid metabolism, hypertension and inflammation. sEH protein levels were 1.8-fold higher in the epididymal fat pad isolated from mice that received a HFD vs a SFD (p>0.05). In order to further evaluate this difference, western blot analysis for sEH was performed on total protein extracted from the epididymal fat pads from the C57BL/6J mice that received either the HFD or SFD for 20 weeks (n=5). While sEH was easily detectable in the adipose tissue extracts, no difference in sEH protein levels was detected when normalized to β-actin. Similarly, real-time quantitative PCR(RT-QPCR) revealed no difference in sEH mRNA levels in the epididymal fat pads.

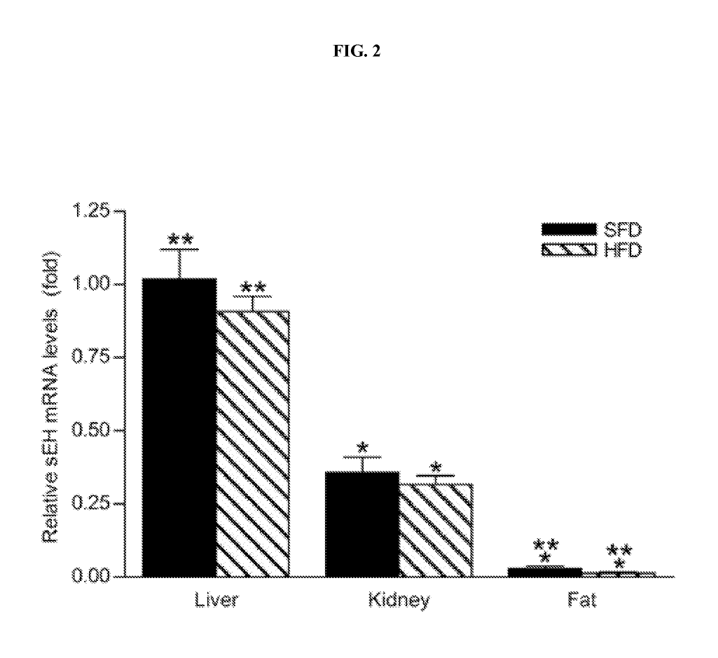

It has been established that sEH is expressed in a variety of other organs, with higher levels found in liver and kidney (Enayetallah et al. (2004) J. Histochem. Cytochem. 52:447-454; Yu et al. (2004) Am. J. Physiol. Renal Physiol. 286:F720-726; Pacifici et al. (1988) Arch. Toxicol. 62:254-257; each herein incorporated by reference in its entirety). RT-QPCR analysis of sEH mRNA levels in liver, kidney and epididymal fat pad of animals fed a SFD or HFD indicated that expression levels differ significantly among organs, with liver>kidney>fat (p<0.0001; FIG. 2). However, diet did not influence the sEH mRNA levels (p>0.05 for each organ) (FIG. 2). To evaluate expression of sEH in different adipose tissue depots, sEH mRNA levels in epididymal fat were compared with those in subcutaneous, perirenal and pericardial fat pads from C57BL/6J mice fed a HFD or a SFD for 13 weeks (n=5) (Table 3). No differences were observed for sEH levels in the different fat depots in comparison with the epididymal fat depot when considering animals fed a HFD; for the animals fed a SFD, perirenal and pericardial sEH mRNA levels were respectively higher and lower than those in the epididymal fat pad. Diet only influenced the sEH mRNA levels in the perirenal fat pad (Table 3). Due to limited amounts of the different fat pads, further analysis focused on the epididymal fat depot.

TABLE 3
Body weight and sEH mRNA levels in different fat pads.

| | SFD | HFD |
|---|---|---|
| Initial weight (g) | 21.3 ± 0.5 | 21.1 ± 0.6 |
| End weight (g)* | 28.9 ± 0.6 | 42.6 ± 0.7 |
| Epididymal fat | 1.15 ± 0.30 | 0.54 ± 0.09 |
| Subcutaneous fat | 0.46 ± 0.07 | 0.77 ± 0.16 |
| Perirenal fat* | 2.14 ± 0.41^{†} | 0.78 ± 0.15 |
| Pericardial fat | 0.02 ± 0.004^{†} | 0.09 ± 0.04 |

Data are mean ± s.e.m. sEH mRNA levels are expressed relative to epididymal fat SFD. sEH, soluble epoxide hydrolase; SFD, standard fat diet; HFD, high fat diet.
*P < 0.001 for SFD vs. HFD;
^{†}P < 0.05 vs. epididymal fat.

### Total Adipose sEH Activity is Increased in Obese Mice

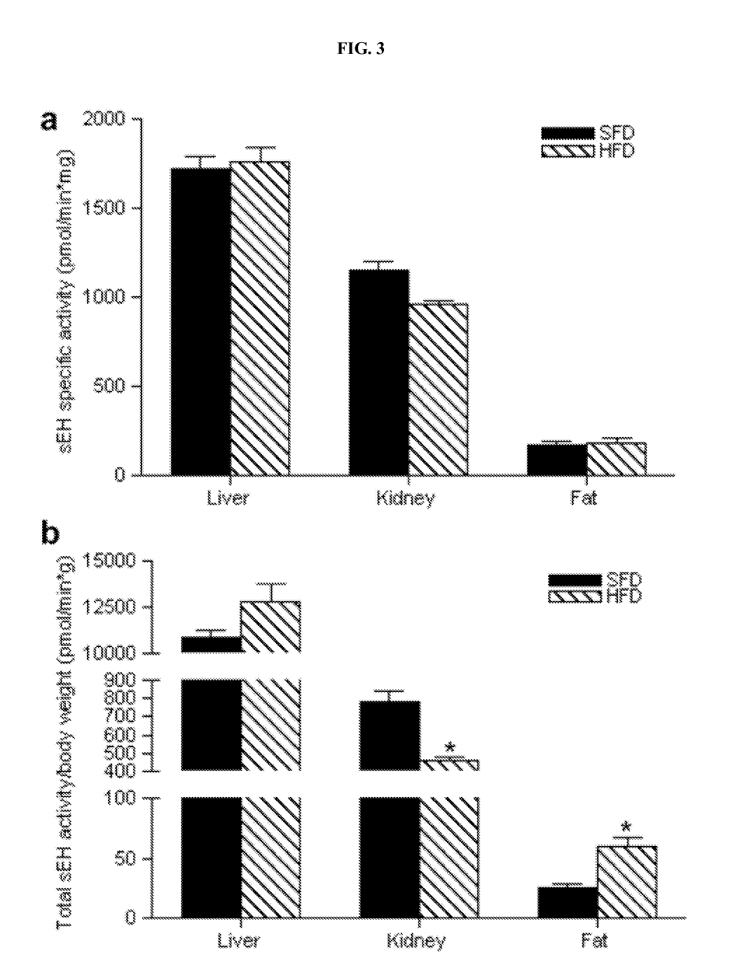

While normalized sEH mRNA and protein levels did not differ in the epididymal fat pad from lean vs obese animals, a higher total adipose sEH activity was anticipated for the obese animals. sEH specific activity was generally in agreement with sEH mRNA data (FIG. 3a). While specific activity differed among organs with liver>kidney>fat (p<0.0001), a HFD diet did not influence the activity. However, when total sEH activity was calculated for each of the different organs and normalized for body weight, the HFD was associated with a 18% increase and a 42% decrease in liver (p=0.10) and kidney (p=0.003) sEH activity, respectively, while it increased total epididymal fat sEH activity by 138% (p=0.008) (FIG. 3b).

### Adipocytes are a Source of sEH

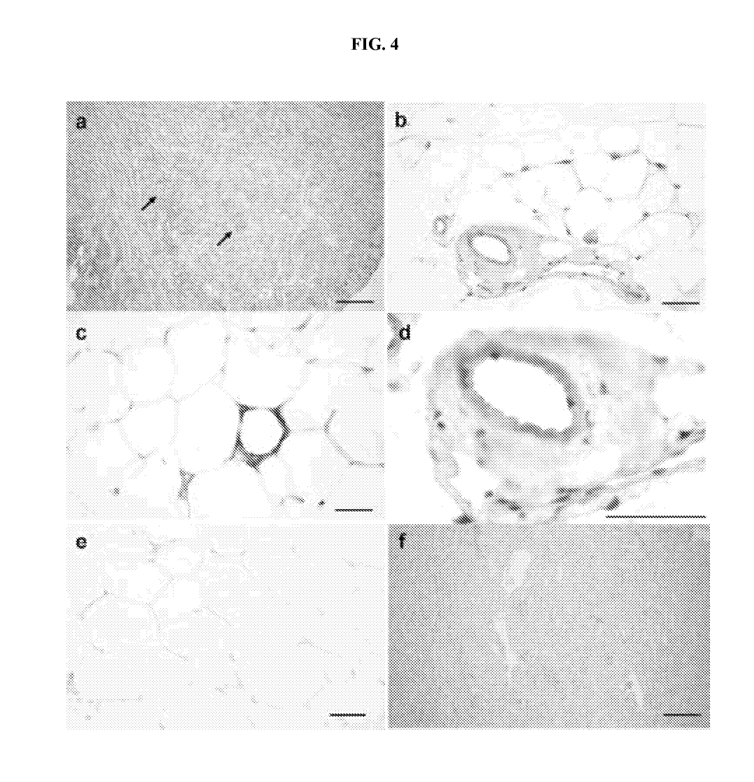

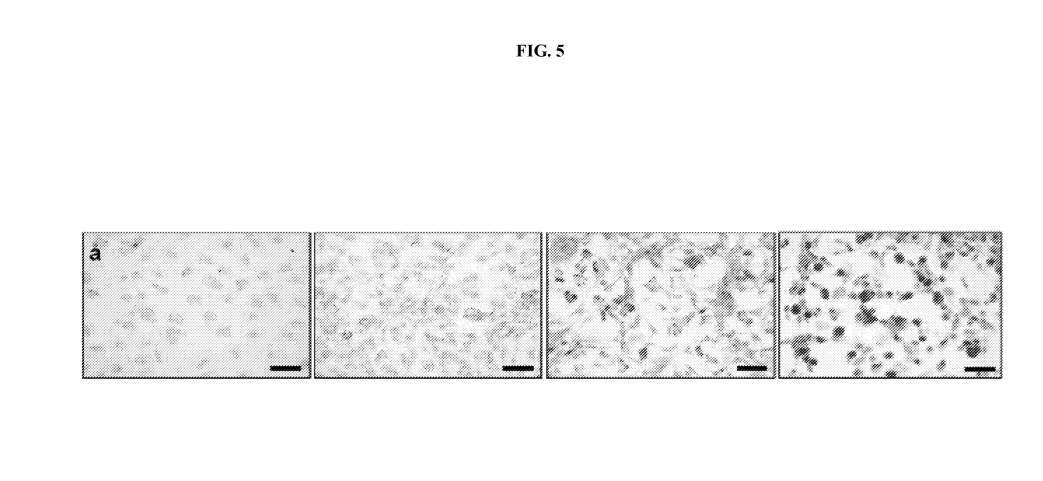

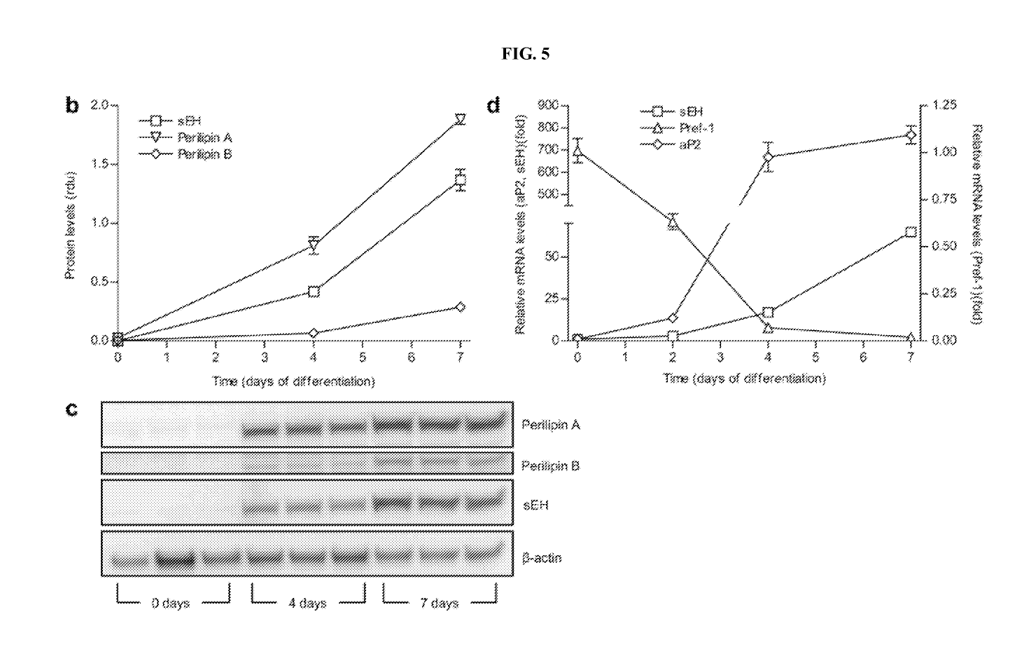

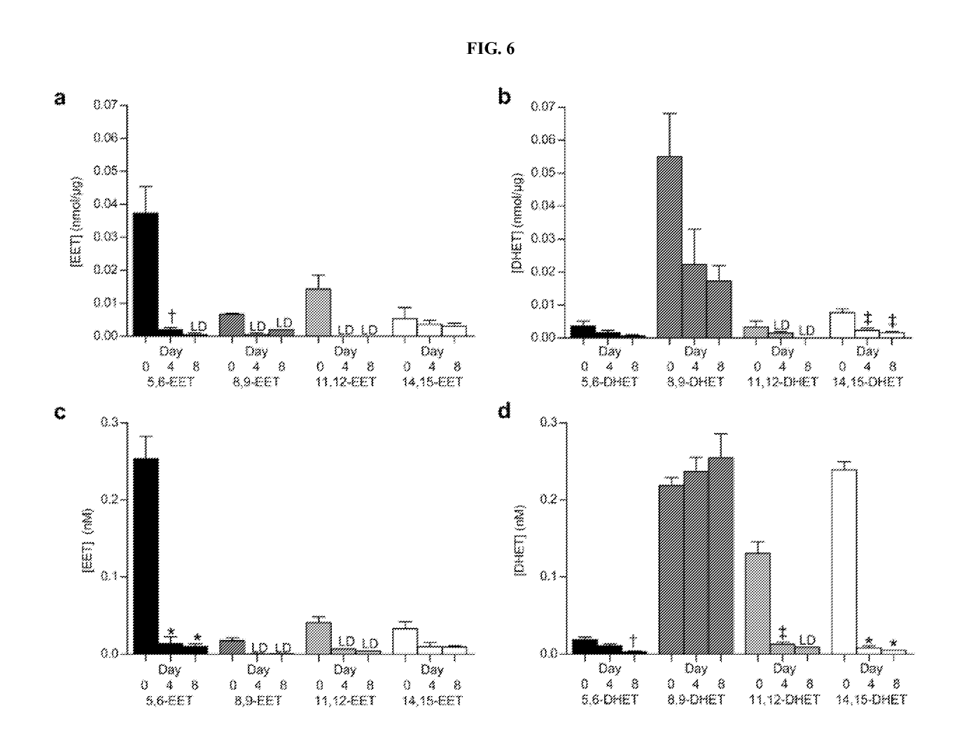

Adipose tissue consists of adipocytes and stromal cells such as endothelial cells and macrophages. Both endothelial cells and macrophages have been shown to express sEH (Yu et al. (2004) Am. J. Physiol. Renal Physiol. 286:F720-426; Rodriguez et al. (2006) Ann. NY Acad. Sci. 1079:130-134; Ai et al. (2007) PNAS 104:9018-9023; each herein incorporated by reference in its entirety). Immunohistochemical analysis of the epididymal fat pads from mice that received either a SFD or HFD for 20 weeks confirmed the presence of sEH in endothelial cells and macrophages in adipose tissue and suggested sEH production in adipocytes (FIG. 4).

After separation of the adipocyte and stromal fractions from the freshly isolated epididymal fat pad from 6-week old C57BL/6J mice fed a SFD, RT-QPCR analysis indicated that both fractions express sEH. In the adipocyte-enriched fraction sEH mRNA levels were 4-fold higher in comparison with the stromal fraction (4.03±0.33 vs 1.01±0.08, p=0.0009, relative to stromal fraction; n=3 per group), indicating that adipocytes actually express sEH. Leptin mRNA and F4/80 mRNA levels were 3.57±0.17 and 1.43±0.06-fold higher in the adipocyte and stromal fractions, respectively, indicative for an enrichment of each fraction (p=0.0002 and 0.04, respectively). The same analysis was performed on freshly isolated epididymal fat pads from C57BL/6J mice fed a HFD for 13 weeks; sEH mRNA levels were again 4-fold higher in the adipocyte-enriched fraction in comparison with the stromal fraction (4.3±0.19 vs 1.05±0.21, p<0.0001, relative to stromal fraction; n=4 per group). Leptin mRNA and F4/80 mRNA levels were 2.56±0.23 and 1.83±0.17-fold higher in the adipocyte and stromal fractions, respectively (p=0.0005 and 0.005, respectively).

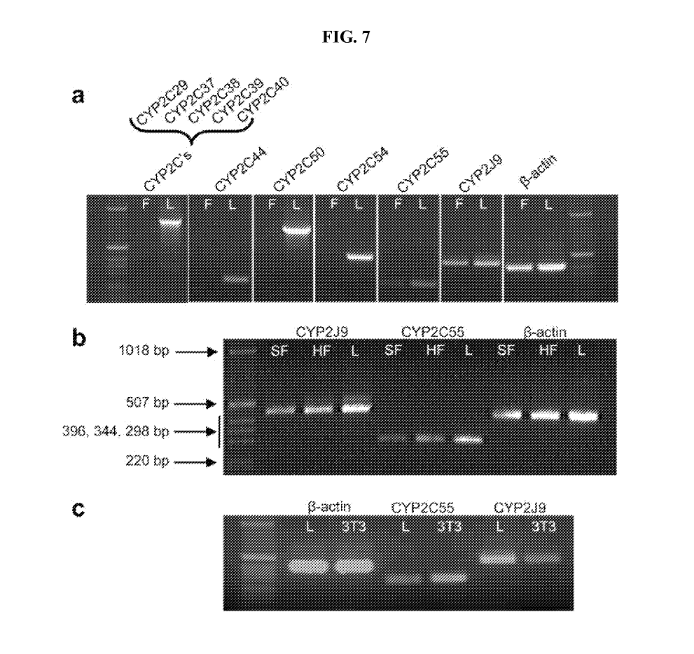

To further characterize sEH expression in adipocytes, 3T3-L1 cells were used in in vitro experiments. sEH was found to be expressed in 3T3-L1 preadipocytes and levels increased significantly upon differentiation into mature adipocytes (FIG. 5). mRNA levels of sEH increased during a 7 day differentiation period (p<0.0001), during which markers of adipogenesis showed an expected profile: fatty acid binding protein (aP2) mRNA increased and preadipocyte factor-1 (Pref-1) mRNA decreased (p<0.0001 for each; FIG. 5d). Lipid accumulation was confirmed by oil red O staining (FIG. 5a). sEH protein levels also increased significantly upon differentiation of 3T3-L1 preadipocytes (p<0.0001; FIG. 5b and c). The increase in perilipin A and B protein was in agreement with the gradual increase of lipid droplet formation (p<0.0001 for both targets) (Greenberg et al. (1993) PNAS 90:12035-12039; herein incorporated by reference in its entirety).

In order to confirm that the sEH expression in adipocytes was biologically active, EET levels were systematically quantified. EET levels were significantly higher in preadipocytes than in mature adipocytes (FIG. 6a). Whereas all 4 EET regioisomers were easily detectable in preadipocytes (day 0), by day 4 the levels had decreased significantly, often below the level of detection (LD). EET levels in cell culture medium showed a profile similar as seen for intracellular EETs (FIG. 6c). Generally, levels of DHETs followed the same trend as EET levels (FIG. 6b and d).

To investigate whether CYP450 enzymes shown to produce the different EET regioisomers from arachidonic acid were present in adipose tissue and mature 3T3-L1 adipocytes, reverse-transcriptase was used for various CYP2C's and CYP2J9 using primers and cycling conditions as described before (Wang et al. (2004) Mol. Pharmacol. 65:1148-1158; Tsao et al. (2000) Mol. Pharmacol. 58:279-287; Qu et al. (2001) J. Biol. Chem. 276:25467-25479; DeLozier et al. (2004) J. Pharmacol. Exp. Ther. 310:845-854; each herein incorporated by reference in its entirety). Interestingly, CYP2C55 and 2J9 were identified in both adipose tissue and mature 3T3-L1 adipocytes (FIG. 7).

### PPARγ Agonists Induce sEH Expression in Mature 3T3-L1 Adipocytes

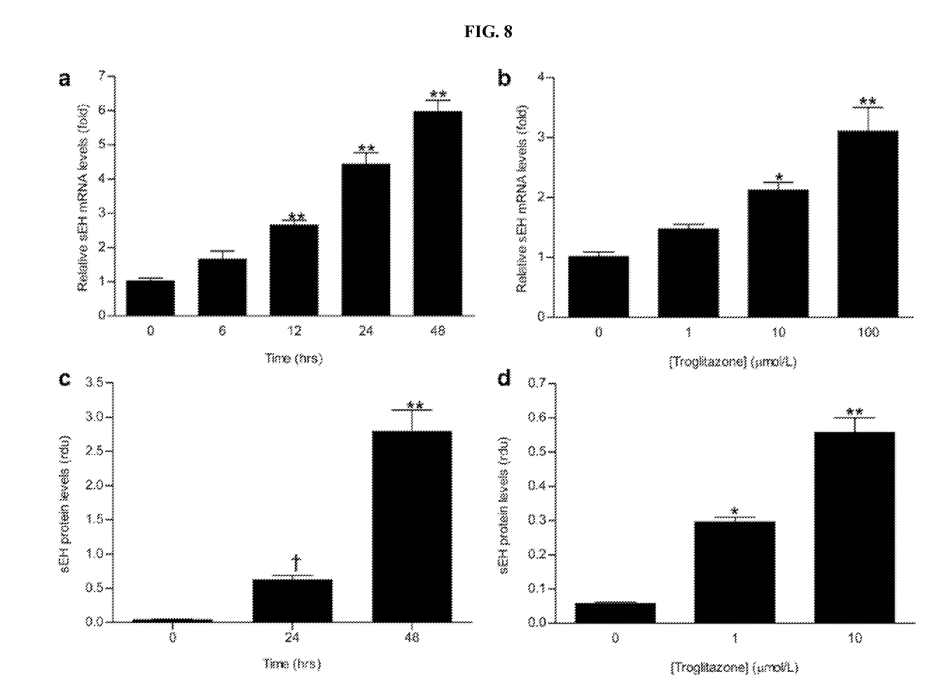

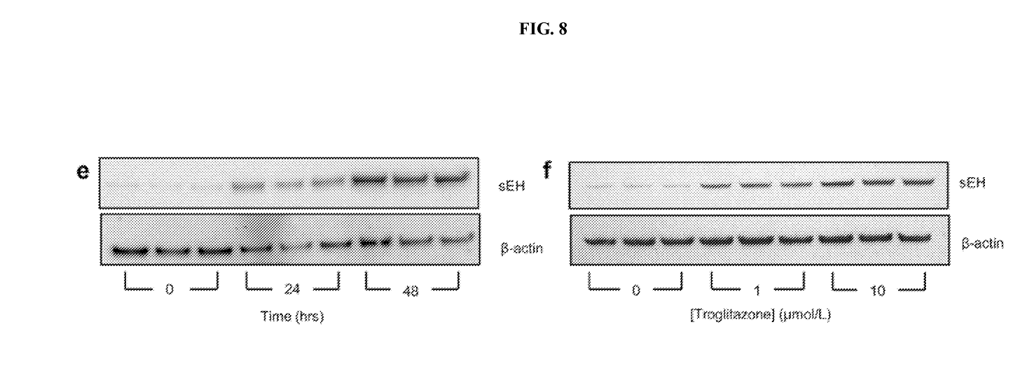

Data presented herein provide evidence that sEH levels increase during adipogenesis. To further examine this relationship, 3T3-L1 adipocytes were treated with troglitazone, a PPARγ agonist known to exert its effect by inducing and maintaining adipogenesis/lipogenesis (Tamori et al. (2002) Diabetes 51:2045-2055; Hamm et al. (1999) 892:134-145; each herein incorporated by reference in its entirety). Treatment of differentiated 3T3-L1 cells with troglitazone resulted in a time-dependent increase in sEH mRNA (FIG. 8a) and protein (FIG. 8c and e). Furthermore, troglitazone exerted a concentration-dependent effect, increasing both sEH mRNA (FIG. 8b) and protein levels (FIG. 8d and f).

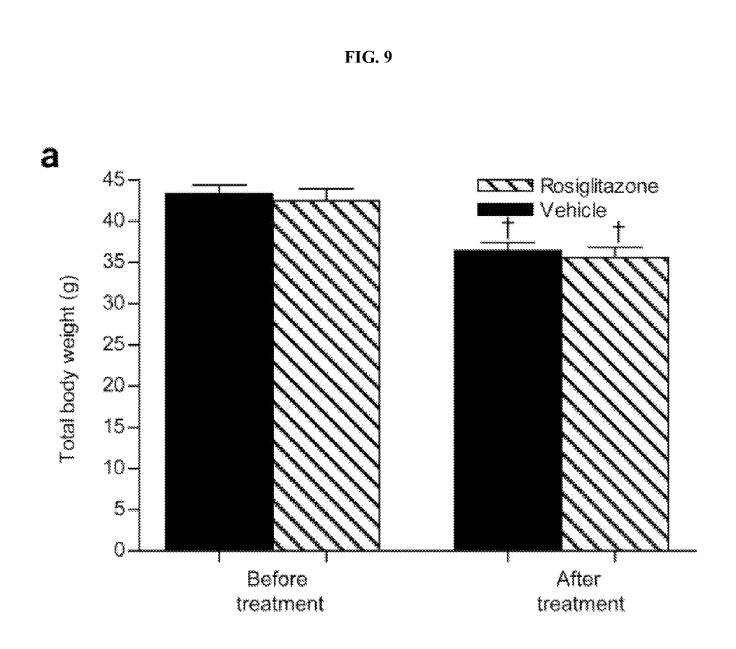

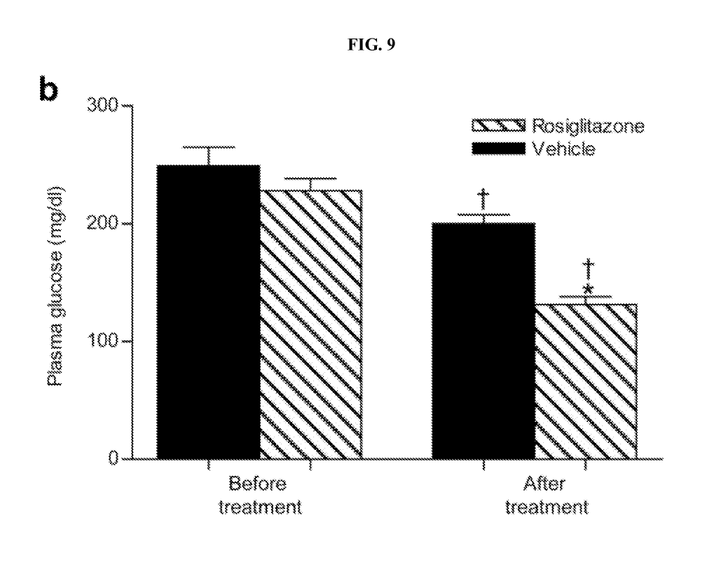

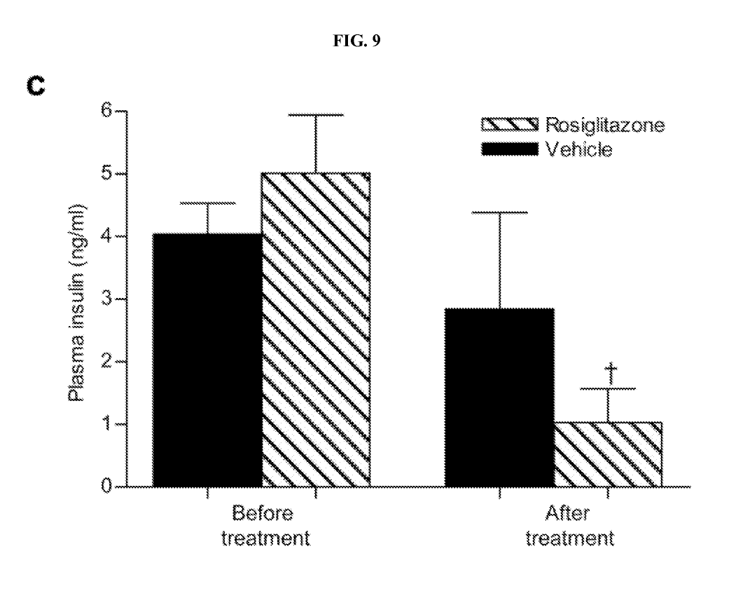

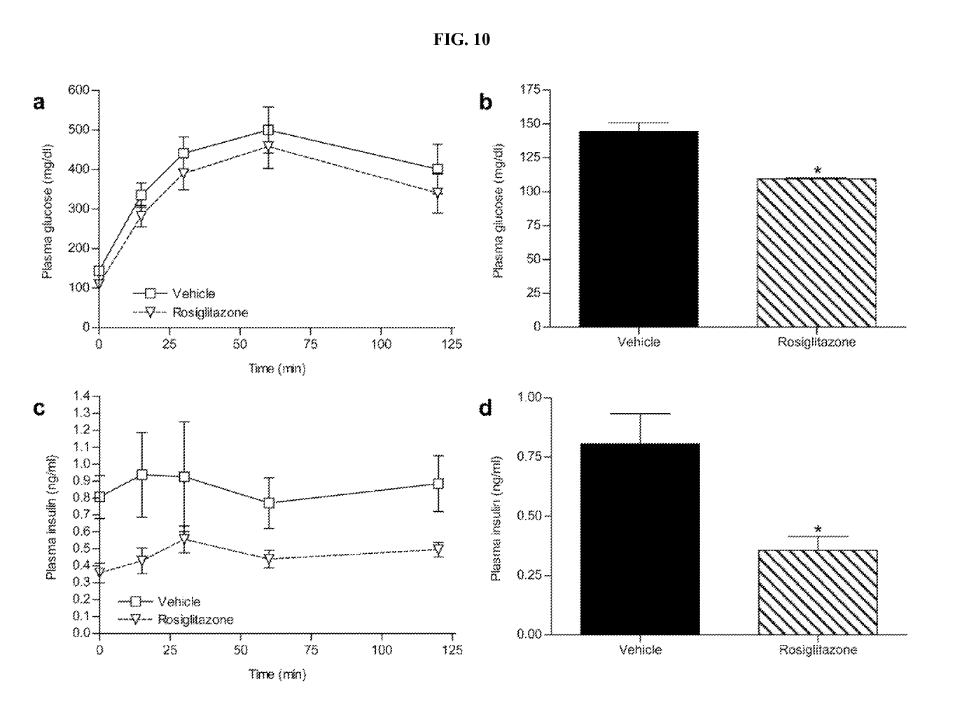

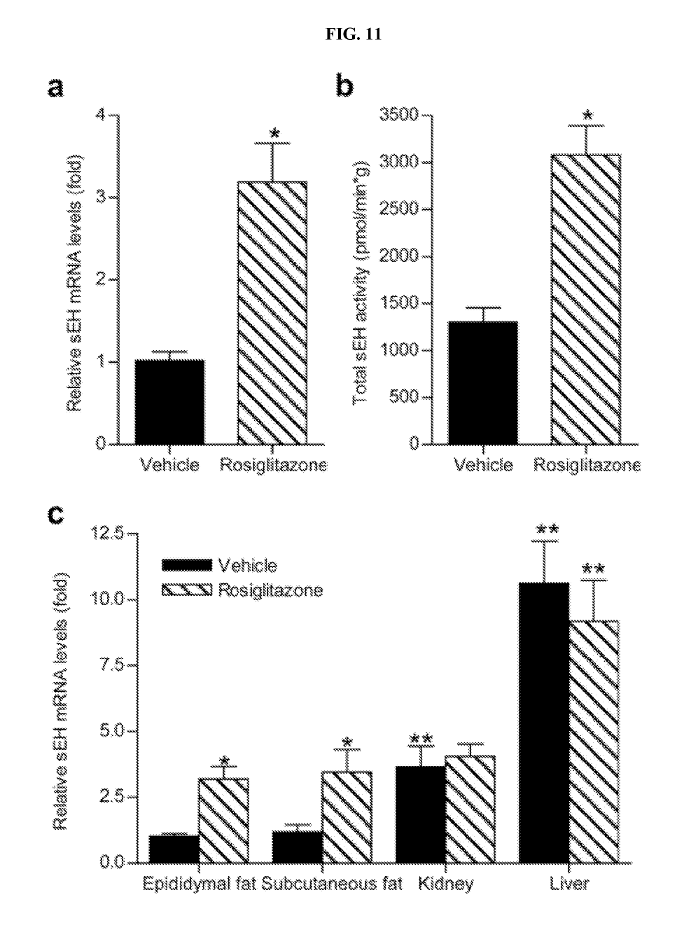

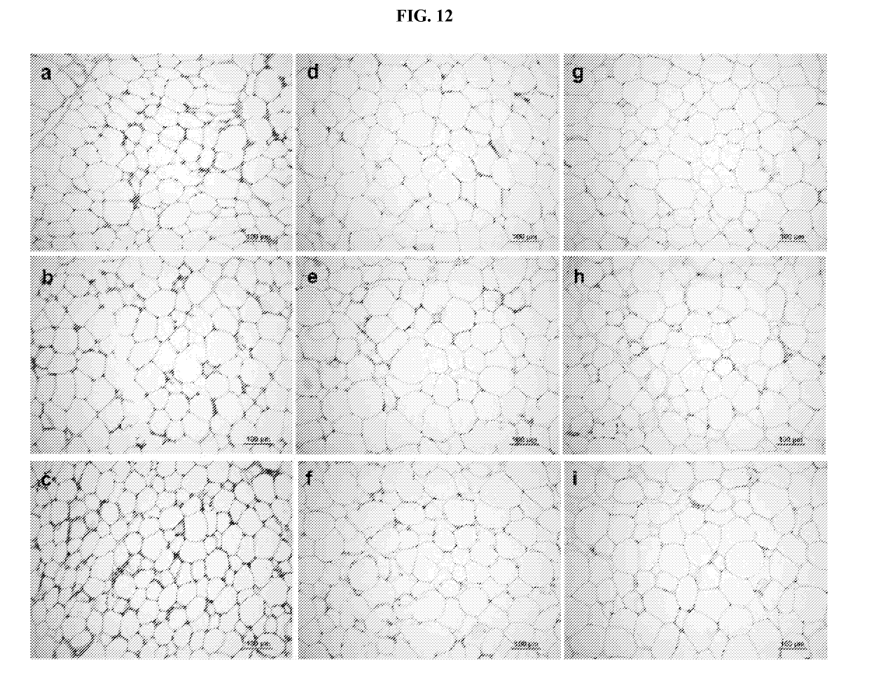

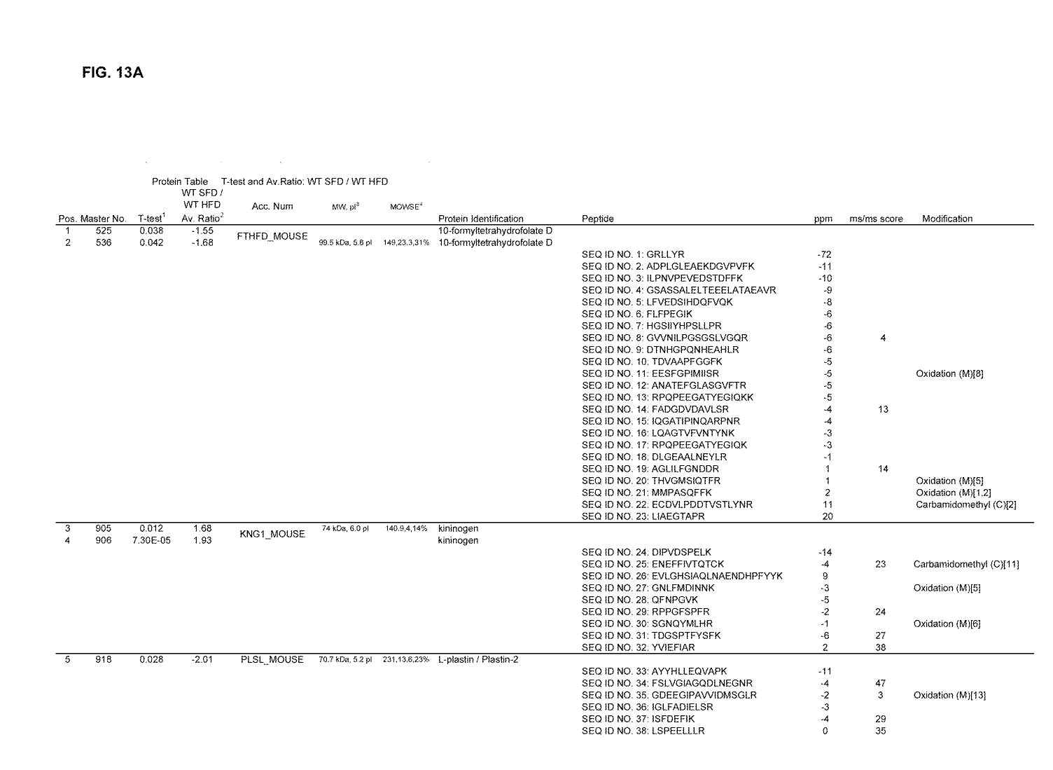

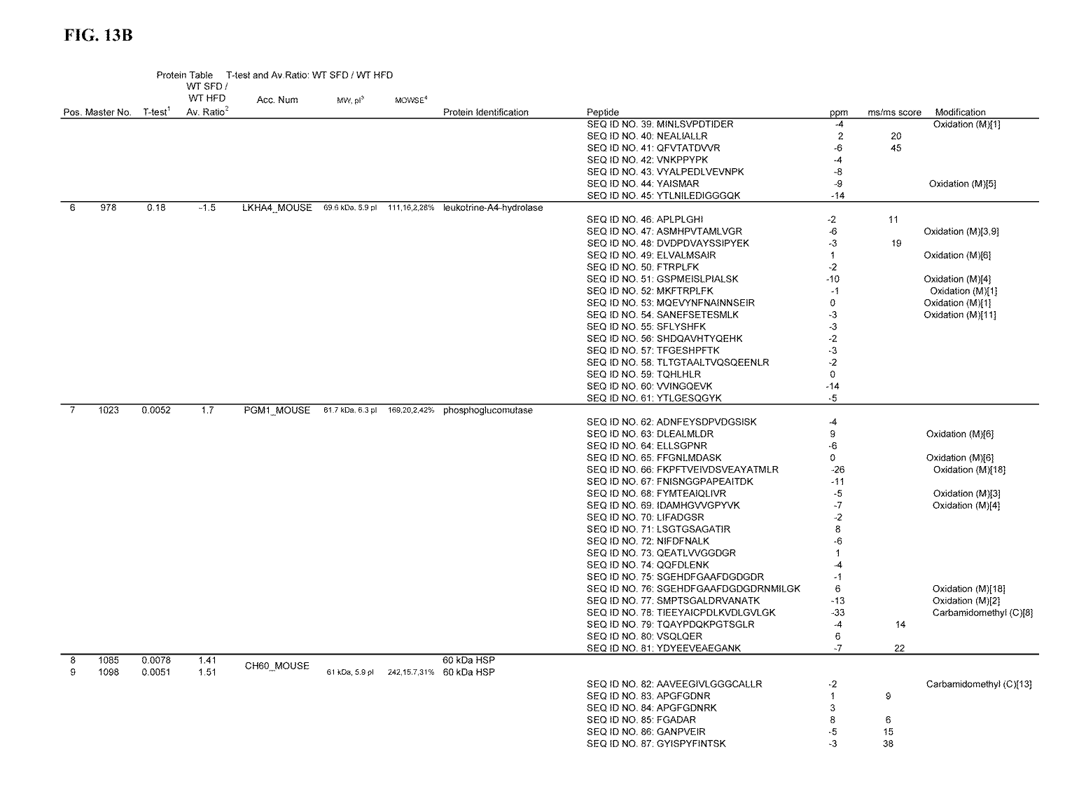

To investigate the effect of PPARγ agonists on sEH expression in adipose tissue in vivo, a diet-induced model of obesity/insulin resistance was used. Obese C57BL/6J mice were treated with 10 mg/kg/day rosiglitazone, a clinically used PPARγ agonist, or vehicle. Data showed that rosiglitazone improved insulin sensitivity, associated with lower plasma insulin and glucose levels, as described before (Liu et al. (2007) Am. J. Physiol. Gastrointest. Liver Physiol. 292:G1671-1682; herein incorporated by reference in its entirety) (FIGS. 9 and 10). sEH mRNA levels and activity were significantly increased in the epididymal fat pad of mice receiving the PPARγ agonist (FIG. 11a and b). Immunohistochemistry indicated that in agreement with mRNA, sEH protein levels were increased in the epididymal fat pad after rosiglitazone treatment (FIG. 12). Whereas sEH mRNA levels in the subcutaneous fat pad showed a profile similar to the epididymal fat pad levels (p=0.03; FIG. 11c), rosiglitazone did not influence liver or kidney sEH mRNA levels (FIG. 11c)). Interestingly, rosiglitazone-induced sEH mRNA levels in the epididymal and subcutaneous fat were similar to kidney sEH mRNA levels (FIG. 11c).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, physiology, biochemistry, medical science, or related fields are intended to be within the scope of the following claims.

1. A method for treating a disease related to diabetes in a subject comprising administration of a peroxisome proliferator-activated receptor gamma agonist and an inhibitor of soluble epoxide hydrolase to said subject.

2. The method of claim 1, wherein said peroxisome proliferator-activated receptor gamma agonist is selected from the group consisting of rosiglitazone, pioglitazone, and troglitazone.

3. The method of claim 1, wherein said inhibitor of soluble hydroxide hydrolase is selected from the group consisting of 1,3-disubstituted ureas and sulfonyl isonipectotamides.

4. The method of claim 3, wherein said 1,3-disubstituted ureas are selected from the group consisting of APAU, AEPU, AUDA, TCUB, TPAU, TUPS, t-ACUB, and ACUB.

5. The method of claim 1, wherein said peroxisome proliferator-activated receptor gamma agonist and said inhibitor of soluble epoxide hydrolase are administered in pharmaceutically acceptable formulations.

6. The method of claim 1, wherein said peroxisome proliferator-activated receptor gamma agonist and said inhibitor of soluble epoxide hydrolase are administered simultaneously.

7. The method of claim 6, wherein said peroxisome proliferator-activated receptor gamma agonist and said inhibitor of soluble epoxide hydrolase are formulated for said simultaneous administration in a single delivery agent.

8. The method of claim 7, wherein said single delivery agent is formulated for oral administration.

9. The method of claim 1, wherein said peroxisome proliferator-activated receptor gamma agonist and said inhibitor of soluble epoxide hydrolase are administered sequentially.

10. The method of claim 1, wherein said disease related to diabetes is selected from the group consisting of diabetes mellitus, type I diabetes, type II diabetes, gestational diabetes, metabolic syndrome, metabolic syndrome X, syndrome X, insulin resistance syndrome, Reaven's syndrome, CHAOS, and malnutrition-related diabetes mellitus.

11. The method of claim 1, wherein said subject is human.

12. The method of claim 1, wherein said subject suffers from edema.

13. The method of claim 1, wherein said subject is at risk for edema.

14. The method of claim 1, wherein said subject suffers from congestive heart failure.

15. The method of claim 1, wherein said subject is at risk for congestive heart failure.

16. A composition comprising a first amount of a peroxisome proliferator-activated receptor gamma agonist and a second amount of an inhibitor of soluble epoxide hydrolase, wherein the first amount and second amount together comprise a therapeutically effective amount of the peroxisome proliferator-activated receptor gamma agonist and the inhibitor of soluble epoxide hydrolase for use as a medicament.

17. The composition of claim 16, wherein said composition is a pharmaceutically acceptable formulation.
